(19) United States
(12) Patent Application Publication
(10) Pub. No.: US 2008/0000032 A1
(43) Pub. Date: Jan. 3, 2008

(54) Use of Metal Complexes Having Bispyridylpyrimidine or Bispyridyltriazine Ligands as Catalysts for Reactions With Peroxy Compounds for Bleaching Coloured Stains on Hard Surfaces

(76) Inventors: Torsten Wieprecht, Schopfheim (DE); Marie-Josee Dubs, Wittersdorf (FR); Gunther Schlingloff, Riehen (CH)

Correspondence Address: CIBA SPECIALTY CHEMICALS CORPORATION;PATENT DEPARTMENT, 540 WHITE PLAINS RD, P O BOX 2005, TARRYTOWN, NY, 10591-9005, US

(21) Appl. No.: 11/587,471

(22) PCT Filed: Apr. 20, 2005
(86) PCT No.: PCT/EP05/51732

§ 371 Date: Oct. 25, 2006

(30) Foreign Application Priority Data
Apr. 29, 2004 (EP) 04101821.9

(51) Int. Cl. B01J 31/18; C07D 213/00; C07D 239/00; C07D 401/14; C07F 13/00; C11D 11/00; C11D 11/02; C11D 17/00; C11D 17/06; C11D 3/39; C11D 3/395

(52) U.S. Cl. 8/111; 510/376

(57) ABSTRACT

The present invention relates to the use of certain manganese complexes having bispyridylpyrimidine or bispyridyltriazine ligands or mixtures of such ligands as catalysts for reactions with peroxy compounds for bleaching coloured stains on hard surfaces, especially dishes in automatic dishwashers. The invention relates also to cleaning formulations for hard surfaces comprising such catalysts.

The present invention relates to the use of certain manganese complexes having bispyridylpyrimidine or bispyridyltriazine ligands or mixtures of such ligands as catalysts for reactions with peroxy compounds for bleaching coloured stains on hard surfaces, especially dishes in automatic dishwashers. The invention also relates to cleaning formulations for hard surfaces comprising such catalysts.

Inorganic peroxy compounds, especially hydrogen peroxide and solid peroxy compounds that dissolve in water with the release of hydrogen peroxide, such as sodium perborate and sodium carbonate perhydrate, have long been used as oxidising agents for disinfection and bleaching purposes. The oxidising action of such substances in dilute solutions is highly dependent upon temperature. For example, using H₂O₂ or perborate in alkaline bleaching liquors it is only at temperatures above about 80° C. that sufficiently rapid bleaching of soiled hard surfaces is achieved. At lower temperatures, the oxidising action of the inorganic peroxy compounds can be improved by the addition of so-called bleach activators, for which numerous proposals have been disclosed in the literature. They are especially compounds from the substance classes of the N- and O-acyl compounds, for example polyacylated alkylenediamines, especially tetraacetylethylenediamine, acylated glycolurils, especially tetraacetylglycoluril, N-acylated hydantoins, hydrazides, triazoles, hydrotriazines, urazoles, diketopiperazines, sulfurylamides and cyanurates, and in addition carboxylic acid anhydrides, especially phthalic anhydride, carboxylic acid esters, especially sodium nonanoyloxybenzenesulfonate, sodium isononanoyloxybenzenesulfonate, and acylated sugar derivatives, such as pentaacetylglucose. By the addition of such substances, the bleaching action of aqueous peroxide liquors can be increased to such an extent that even at temperatures of around 60° C. their action is substantially the same as that of the peroxide liquor alone at 95° C.

Those temperatures are still too high for cleaning hard surfaces, for example table- and kitchen-ware, by hand and are normally not always achieved even in machine dishwashing methods. In the search for energy-saving methods of cleaning table- and kitchen-ware by machine, in recent years temperatures of less than 60° C., especially less than 50° C., have been gaining importance.

At such low temperatures the action of the previously known activator compounds usually declines noticeably, especially in the case of stains that are difficult to bleach, such as tea residues on porcelain or glass. There has therefore been no lack of efforts to develop activators that are more effective in that temperature range, but without there having been any convincing success to date.

A starting point might be provided by the use of transition metal salts and complexes as so-called bleach catalysts. WO 97/07191 already discloses cleaning formulations for table- and kitchen-ware that comprise transition metal complexes of the salen type as activators for peroxy compounds, but those compounds too are still unable to meet all requirements.

The aim of the present invention was accordingly to provide improved metal complex catalysts for oxidation processes that meet the above requirements and, especially, enhance the action of peroxide compounds in automatic dishwashing machines without causing any appreciable damage.

The addition of complexes of the present invention in catalytic amounts to a dishwashing formulation that comprises a peroxy compound and optionally TAED (N,N,N′,N′-tetraacetylethylenediamine) or other bleach activators results in the substantial removal of tea stains from porcelain in a dishwasher. This is the case even when hard water is used, it being known that tea deposits are more difficult to remove in hard water than in soft water.

The invention accordingly relates to the use of at least one metal complex of formula (1)
[LₙMeₘXₚ]^{z}Y_{q} (1),
wherein
Me is manganese, titanium, iron, cobalt, nickel or copper,
X is a coordinating or bridging radical,
n and m are each independently of the other an integer having a value of from 1 to 8,
p is an integer having a value of from 0 to 32,
z is the charge of the metal complex,
Y is a counter-ion,
q=z/(charge of Y), and
L is a ligand of formula (2) 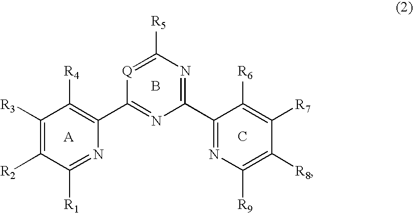
wherein
Q is N or CR₁₀,
R₁, R₂, R₃, R₄, R₅, R₆, R₇, R₈, R₉ and R₁₀ are each independently of the others hydrogen; unsubstituted or substituted C₁-C₁₈alkyl or unsubstituted or substituted aryl; cyano; halogen; nitro; —COOR₁₁ or —SO₃R₁₁ wherein
R₁₁ is in each case hydrogen, a cation or unsubstituted or substituted C₁-C₁₈alkyl or unsubstituted or substituted aryl;
   —SR₁₂, —SO₂R₁₂ or —OR₁₂ wherein
R₁₂ is in each case hydrogen or unsubstituted or substituted C₁-C₁₈alkyl or unsubstituted or substituted aryl;
   —NR₁₃R₁₄; —(C₁-C₆alkylene)-NR₁₃R₁₄; —N^{⊕}R₁₃R₁₄R₁₅; —(C₁-C₆alkylene)-N^{⊕}R₁₃R₁₄R₁₅; —N(R₁₂)—(C₁-C₆alkylene)-NR₁₃R₁₄; —N[(C₁-C₆alkylene)-NR₁₃R₁₄]₂; —N(R₁₂)—(C₁-C₆alkylene)-N^{⊕}R₁₃R₁₄R₁₅; —N[(C₁-C₆alkylene)-N^{⊕}R₁₃R₁₄R₁₅]₂; —N(R₁₂)—N—R₁₃R₁₄ or —N(R₁₂)—N^{⊕}R₁₃R₁₄R₁₅, wherein
R₁₂ is as defined above and
R₁₃, R₁₄ and R₁₅ are each independently of the other(s) hydrogen or unsubstituted or substituted C₁-C₁₈alkyl or unsubstituted or substituted aryl, or
R₁₃ and R₁₄, together with the nitrogen atom linking them, form an unsubstituted or substituted 5-, 6- or 7-membered ring which may contain further hetero atoms,
   as catalyst(s) for bleach reactions in cleaning formulations for hard surfaces.

Preferably the compounds of formula (1) are used as catalysts for bleach reactions in dishwashing formulations. Such dishwashing formulations are preferably used in automatic dishwashing machines.

Suitable substituents for the alkyl groups, aryl groups, alkylene groups or 5-, 6- or 7-membered rings are especially C₁-C₄alkyl; C₁-C₄alkoxy; hydroxy; sulfo; sulfato; halogen; cyano; nitro; carboxy; amino; N-mono- or N,N-di-C₁-C₄alkylamino unsubstituted or substituted by hydroxy in the alkyl moiety; N-phenylamino; N-naphthylamino; phenyl; phenoxy or naphthyloxy.

The C₁-C₁₈alkyl radicals mentioned for the compounds of formula (2) are, for example, straight-chain or branched alkyl radicals, such as methyl, ethyl, n-propyl, isopropyl, n-butyl, sec-butyl, isobutyl, tert-butyl or straight-chain or branched pentyl, hexyl, heptyl or octyl. Preference is given to C₁-C₁₂alkyl radicals, especially C₁-C₈alkyl radicals and preferably C₁-C₄alkyl radicals. The mentioned alkyl radicals may be unsubstituted or substituted e.g. by hydroxy, C₁-C₄alkoxy, sulfo or by sulfato, especially by hydroxy. The corresponding unsubstituted alkyl radicals are preferred. Very special preference is given to methyl and ethyl, especially methyl.

Examples of aryl radicals that come into consideration for the compounds of formula (2) are phenyl or naphthyl each unsubstituted or substituted by C₁-C₄alkyl, C₁-C₄alkoxy, halogen, cyano, nitro, carboxy, sulfo, hydroxy, amino, N-mono- or N,N-di-C₁-C₄alkylamino unsubstituted or substituted by hydroxy in the alkyl moiety, N-phenylamino, N-naphthylamino, wherein the amino groups may be quaternised, phenyl, phenoxy or by naphthyloxy. Preferred substituents are C₁-C₄alkyl, C₁-C₄alkoxy, phenyl and hydroxy. Special preference is given to the corresponding phenyl radicals.

The C₁-C₆alkylene groups mentioned for the compounds of formula (2) are, for example, straight-chain or branched alkylene radicals, such as methylene, ethylene, n-propylene or n-butylene. C₁-C₄alkylene groups are preferred. The alkylene radicals mentioned may be unsubstituted or substituted, for example by hydroxy or C₁-C₄alkoxy.

In the compounds of formulae (1) and (2), halogen is preferably chlorine, bromine or fluorine, with special preference being given to chlorine.

Examples of cations that come into consideration for compounds of formulae (1) and (2) include alkali metal cations, such as lithium, potassium and especially sodium, alkaline earth metal cations, such as magnesium and calcium, and ammonium cations. The alkali metal cations, especially sodium, are preferred.

Suitable metal ions for Me for the compounds of formula (1) are, for example, manganese in oxidation states II-V, titanium in oxidation states III and IV, iron in oxidation states I to IV, cobalt in oxidation states I to III, nickel in oxidation states I to III and copper in oxidation states I to III, with special preference being given to manganese, especially manganese in oxidation states II to IV, preferably in oxidation state II. Also of interest are titanium IV, iron II-IV, cobalt II-III, nickel II-III and copper II-III, especially iron II-IV.

For the radical X for the compounds of formula (1) there come into consideration, for example, CH₃CN; H₂O; F⁻; Cl⁻; Br⁻; HOO⁻; O₂²⁻; O²⁻; R₁₆COO⁻; R₁₆O⁻; LMeO⁻ and LMeOO⁻, wherein R₁₆ is hydrogen, —SO₃C₁-C₄alkyl or unsubstituted or substituted C₁-C₁₈alkyl or unsubstituted or substituted aryl, and C₁-C₁₈alkyl, aryl, L and Me have the definitions and preferred meanings given hereinabove and herein below. Especially preferably, R₁₆ is hydrogen; C₁-C₄alkyl; sulfophenyl or phenyl, especially hydrogen.

As counter-ion Y for the compounds of formula (1) there come into consideration, for example, R₁₇COO⁻; ClO₄⁻; BF₄⁻; PF₆⁻; R₁₇SO₃⁻; R₁₇SO₄⁻; SO₄²⁻; NO₃⁻; F⁻; Cl⁻, Br⁻ and I⁻, wherein R₁₇ is hydrogen or unsubstituted or substituted C₁-C₁₈alkyl or unsubstituted or substituted aryl. R₁₇ as C₁-C₁₈alkyl or aryl has the definitions and preferred meanings given hereinabove and herein below. Especially preferably, R₁₇ is hydrogen; C₁-C₄alkyl; phenyl or sulfophenyl, especially hydrogen or 4-sulfophenyl. The charge of the counter-ion Y is accordingly preferably 1- or 2-, especially 1-.

Y can also be a customary organic counter-ion, for example citrate, oxalate or tartrate. For the compounds of formula (1), n is preferably an integer having a value of from 1 to 4, preferably 1 or 2 and especially 1.

For the compounds of formula (1), m is preferably an integer having a value of 1 or 2, especially 1.

For the compounds of formula (1), p is preferably an integer having a value of from 0 to 4, especially 2.

For the compounds of formula (1), z is preferably an integer having a value of from 8− to 8+, especially from 4− to 4+ and especially preferably from 0 to 4+. z is more especially the number 0.

For the compounds of formula (1), q is preferably an integer from 0 to 8, especially from 0 to 4, and is especially preferably the number 0.

R₁₁ in compounds of formula (2) is preferably hydrogen, a cation, C₁-C₁₂alkyl, unsubstituted phenyl or phenyl substituted as indicated above. Especially preferably, R₁₁ is hydrogen, an alkali metal cation, alkaline earth metal cation or ammonium cation, C₁-C₄alkyl or phenyl, especially hydrogen or an alkali metal cation, alkaline earth metal cation or ammonium cation.

R₁₂ in compounds of formula (2) is preferably hydrogen, C₁-C₁₂alkyl, unsubstituted phenyl or phenyl substituted as indicated above. Especially preferably, R₁₂ is hydrogen, C₁-C₄alkyl or phenyl, more especially hydrogen or C₁-C₄alkyl, preferably hydrogen. Examples of the radical of formula —OR₁₂ that may be mentioned are hydroxy and C₁-C₄alkoxy, such as methoxy and especially ethoxy.

When R₁₃ and R₁₄ in compounds of formula (2), together with the nitrogen atom linking them, form a 5-, 6- or 7-membered ring, that ring is preferably an unsubstituted or C₁-C₄alkyl-substituted pyrrolidine, piperidine, piperazine, morpholine or azepane ring, wherein the amino groups may be quaternised, in which case preferably the nitrogen atoms that are not bonded directly to one of the three rings A, B or C are quaternised.

The piperazine ring may, for example, be substituted by one or two unsubstituted C₁-C₄alkyl and/or substituted C₁-C₄alkyl at the nitrogen atom not bonded to the pyridine ring. In addition, R₁₃, R₁₄ and R₁₅ are preferably hydrogen, unsubstituted or hydroxy-substituted C₁-C₁₂alkyl, unsubstituted phenyl or phenyl substituted as indicated above. Special preference is given to hydrogen, unsubstituted or hydroxy-substituted C₁-C₄alkyl or unsubstituted or hydroxy-substituted phenyl, especially hydrogen or unsubstituted or hydroxy-substituted C₁-C₄alkyl, preferably hydrogen.

Preference is given to ligands L of formula (2) wherein R₅ is not hydrogen.

R₅ in L of formula (2) is preferably C₁-C₁₂alkyl; phenyl unsubstituted or substituted by C₁-C₄alkyl, C₁-C₄alkoxy, halogen, cyano, nitro, carboxy, sulfo, hydroxy, amino, N-mono- or N,N-di-C₁-C₄alkylamino unsubstituted or substituted by hydroxy in the alkyl moiety, N-phenyl-amino, N-naphthylamino, phenyl, phenoxy or by naphthyloxy; cyano; halogen; nitro; —COOR₁₁ or —SO₃R₁₁ wherein R₁₁ is in each case hydrogen, a cation, C₁-C₁₂alkyl, unsubstituted phenyl or phenyl substituted as indicated above; —SR₁₂, —SO₂R₁₂ or —OR₁₂ wherein R₁₂ is in each case hydrogen, C₁-C₁₂alkyl, unsubstituted phenyl or phenyl substituted as indicated above; —NR₁₃R₁₄; —(C₁-C₆alkylene)-NR₁₃R₁₄; —N^{⊕}R₁₃R₁₄R₁₅; —(C₁-C₆alkylene)-N^{⊕}R₁₃R₁₄R₁₅; —N(R₁₂)—(C₁-C₆alkylene)-NR₁₃R₁₄; —N(R₁₂)—(C₁-C₆alkylene)-N^{⊕}R₁₃R₁₄R₁₅; —N(R₁₂)—N—R₁₃R₁₄ or —N(R₁₂)—N^{⊕}R₁₃R₁₄R₁₅, wherein R₁₂ may have one of the meanings given above and R₁₃, R₁₄ and R₁₅ are each independently of the other(s) hydrogen, unsubstituted or hydroxy-substituted C₁-C₁₂alkyl, unsubstituted phenyl or phenyl substituted as indicated above, or R₁₃ and R₁₄, together with the nitrogen atom linking them, form a pyrrolidine, piperidine, piperazine, morpholine or azepane ring unsubstituted or substituted by at least one unsubstituted C₁-C₄alkyl and/or substituted C₁-C₄alkyl, wherein the nitrogen atom may be quaternised.

R₅ in L of formula (2) is especially preferably phenyl unsubstituted or substituted by C₁-C₄alkyl, C₁-C₄alkoxy, halogen, phenyl or by hydroxy; cyano; nitro; —COOR₁₁ or —SO₃R₁₁ wherein R₁₁ is in each case hydrogen, a cation, C₁-C₄alkyl or phenyl; —SR₁₂, —SO₂R₁₂ or —OR₁₂ wherein R₁₂ is in each case hydrogen, C₁-C₄alkyl or phenyl; —N(CH₃)—NH₂ or —NH—NH₂; amino; N-mono- or N,N-di-C₁-C₄alkylamino unsubstituted or substituted by hydroxy in the alkyl moiety; or an unsubstituted or C₁-C₄alkyl-substituted pyrrolidine, piperidine, piperazine, morpholine or azepane ring.

R₅ in L of formula (2) is very especially preferably C₁-C₄alkoxy; hydroxy; phenyl unsubstituted or substituted by C₁-C₄alkyl, C₁-C₄alkoxy, phenyl or by hydroxy; hydrazine; amino; N-mono- or N,N-di-C₁-C₄alkylamino unsubstituted or substituted by hydroxy in the alkyl moiety; or an unsubstituted or C₁-C₄alkyl-substituted pyrrolidine, piperidine, piperazine, morpholine or azepane ring.

As radicals R₅ in L of formula (2) there are especially important C₁-C₄alkoxy; hydroxy; hydrazine; amino; N-mono- or N,N-di-C₁-C₄alkylamino unsubstituted or substituted by hydroxy in the alkyl moiety; and an unsubstituted or C₁-C₄alkyl-substituted pyrrolidine, piperidine, piperazine, morpholine or azepane ring.

As radicals R₅ in L of formula (2) there are very especially important C₁-C₄alkoxy; hydroxy; N-mono- or N,N-di-C₁-C₄alkylamino substituted by hydroxy in the alkyl moiety; and an unsubstituted or C₁-C₄alkyl-substituted pyrrolidine, piperidine, piperazine, morpholine or azepane ring. Of those, hydroxy is of special interest.

The preferred meanings given above for R₅ apply also to R₁, R₂, R₃, R₄, R₆, R₇, R₈, R₉ and R₁₀ in L of formula (2), but those radicals may additionally be hydrogen.

According to one embodiment of the present invention, R₁, R₂, R₃, R₄, R₆, R₇, R₈, R₉ and R₁₀ in L of formula (2) are hydrogen and R₅ in L of formula (2) is a radical other than hydrogen having the definition and preferred meanings indicated above.

According to a further embodiment of the present invention, R₁, R₂, R₄, R₆, R₈, R₉ and R₁₀ in L of formula (2) are hydrogen and R₃, R₅ and R₇ in L of formula (2) are radicals other than hydrogen, for each of which the definition and preferred meanings indicated above for R₅ apply.

Preferred as ligands L are those of formula (3a) and/or (3b) 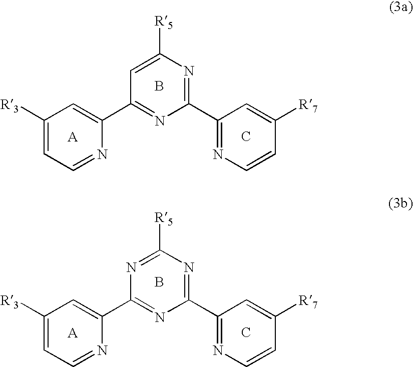
wherein R′₃ and R′₇ have the definitions and preferred meanings indicated above for R₃ and R₇, and R′₅ has the definition and preferred meanings indicated above for R₅.

A preferred embodiment of the present invention relates to the use of at least one Mn(II)-complex of formula (3c) and/or (3d) 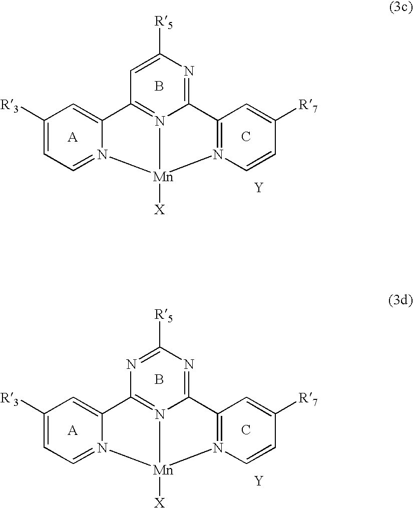
wherein
R′₅ is hydroxy; N-mono- or N,N-di-C₁-C₂alkylamino unsubstituted or substituted by hydroxy in the alkyl moiety; or —NR₁₃R₁₄; —(C₁-C₂alkylene)-NR₁₃R₁₄; —N(R₁₂)—(C₁-C₂alkylene)-NR₁₃R₁₄; —N[(C₁-C₂alkylene)-NR₁₃R₁₄]₂; or —N(R₁₂)—N—R₁₃R₁₄, wherein
   R₁₂ is hydrogen; C₁-C₄alkyl or unsubstituted phenyl or phenyl substituted by (substituted in the alkyl moiety by hydroxy) N-mono- or N,N-di-C₁-C₂alkylamino-, N-phenylamino-, N-naphthylamino-, phenyl-, phenoxy- or naphthyloxy, and
   R₁₃ and R₁₄ are each independently of the other hydrogen, unsubstituted or hydroxy-substituted C₁-C₄alkyl, unsubstituted phenyl or phenyl substituted as indicated above, or
   R₁₃ and R₁₄, together with the nitrogen atom linking them, form a pyrrolidine, piperidine, piperazine, morpholine or azepane ring that is unsubstituted or substituted by at least one unsubstituted C₁-C₄alkyl and/or substituted C₁-C₄alkyl, especially a pyrrolidine, piperidine, piperazine, morpholine or azepane ring, and
R′₃ and R′₇ are each independently of the other hydrogen; halogen; hydroxy; N-mono- or N,N-di-C₁-C₂alkylamino substituted by hydroxy in the alkyl moiety; or —NR₁₃R₁₄; —(C₁-C₂alkylene)-NR₁₃R₁₄; —N(R₁₂)—(C₁-C₂alkylene)-NR₁₃R₁₄; —N[(C₁-C₂alkylene)-NR₁₃R₁₄]₂; or —N(R₁₂)—N—R₁₃R₁₄, wherein
   R₁₂ is hydrogen; C₁-C₄alkyl or unsubstituted phenyl or phenyl substituted by (substituted in the alkyl moiety by hydroxy) N-mono- or N,N-di-C₁-C₂alkylamino-, N-phenylamino-, N-naphthylamino-, phenyl-, phenoxy- or naphthyloxy, and
   R₁₃ and R₁₄ are each independently of the other hydrogen; unsubstituted or hydroxy-substituted C₁-C₄alkyl, unsubstituted phenyl or phenyl substituted as indicated above, or
   R₁₃ and R₁₄, together with the nitrogen atom linking them, form a pyrrolidine, piperidine, piperazine, morpholine or azepane ring that is unsubstituted or substituted by at least one unsubstituted C₁-C₄alkyl and/or substituted C₁-C₄alkyl, especially a pyrrolidine, piperidine, piperazine, morpholine or azepane ring,
      X is F⁻; Cl⁻; Br⁻; HOO⁻; ⁻CH₃COO⁻; HCOO⁻ or HO⁻, and
      Y is CH₃COO⁻; HCOO⁻; ClO₄⁻; BF₄⁻; PF₆⁻; HSO₃⁻; HSO₄⁻; NO₃⁻; F⁻; Cl⁻; Br⁻ or I⁻.

A more preferred embodiment of the present invention relates to the use of at least one Mn(II)-complex of formula (3c) and/or (3d) 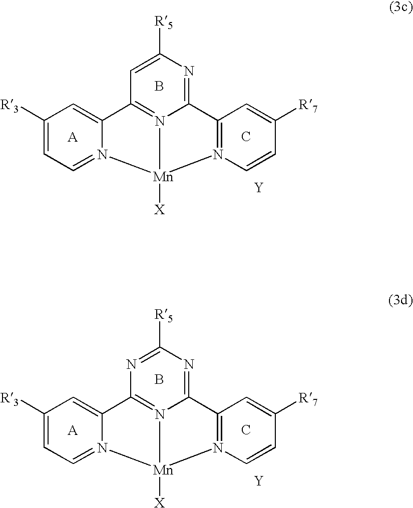
wherein
R′₅ is hydroxy; N-mono- or N,N-di-C₁-C₂alkylamino unsubstituted or substituted by hydroxy in the alkyl moiety; or —NH₂
   R₁₃ and R₁₄ are each independently of the other hydrogen, unsubstituted or
R₁₃ and R₁₇ are each independently of the other hydrogen; Cl; hydroxy; N-mono- or N,N-di-C₁-C₂alkylamino substituted by hydroxy in the alkyl moiety; 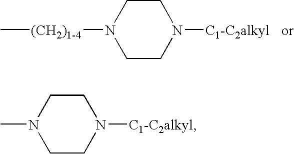
X is F⁻; Cl⁻; Br⁻; HOO⁻; CH₃COO⁻; HCOO⁻ or HO⁻, and
Y is CH₃COO⁻; HCOO⁻; ClO₄⁻; BF₄⁻; PF₆⁻; HSO₃⁻; HSO₄⁻; NO₃⁻; F⁻; Cl⁻; Br⁻ or I⁻.

An embodiment of the invention to which preference is likewise given is the use of at least one metal complex compound of formula (1′)
[L′ₙMeₘXₚ]^{z}Y_{q} (1′),
wherein
Me is manganese, titanium, iron, cobalt, nickel or copper,
X is a coordinating or bridging radical,
n and m are each independently of the other an integer having a value of from 1 to 8,
p is an integer having a value of from 0 to 32,
z is the charge of the metal complex,
Y is a counter-ion,
q=z/(charge of Y), and
L′ is a ligand of formula (2′) 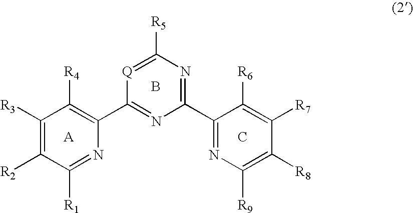
wherein
Q is N or CR₁₀,
R₁, R₂, R₃, R₄, R₅, R₆, R₇, R₈, R₉ and R₁₀ are each independently of the others hydrogen; unsubstituted or substituted C₁-C₁₈alkyl or unsubstituted or substituted aryl; cyano; halogen; nitro; —COOR₁, or —SO₃R₁₁ wherein
R₁₁ is in each case hydrogen, a cation or unsubstituted or substituted C₁-C₁₈alkyl or unsubstituted or substituted aryl;
   —SR₁₂, —SO₂R₁₂ or —OR₁₂ wherein
R₁₂ is in each case hydrogen or unsubstituted or substituted C₁-C₁₈alkyl or unsubstituted or substituted aryl;
   —NR₁₃R₁₄; —(C₁-C₆alkylene)-NR₁₃R₁₄; —N^{⊕}R₁₃R₁₄R₁₅; —(C₁-C₆alkylene)-N^{⊕}R₁₃R₁₄R₁₅; —N(R₁₂)—(C₁-C₆alkylene)-NR₁₃R₁₄; —N[(C₁-C₆alkylene)-NR₁₃R₁₄]₂; —N(R₁₂)—(C₁-C₆alkylene)-N^{⊕}R₁₃R₁₄R₁₅; —N[(C₁-C₆alkylene)-N^{⊕}R₁₃R₁₄R₁₅]₂; —N(R₁₂)—N—R₁₃R₁₄ or —N(R₁₂)—N^{⊕}R₁₃R₁₄R₁₅, wherein
R₁₂ is as defined above and
R₁₃, R₁₄ and R₁₅ are each independently of the other(s) hydrogen or unsubstituted or substituted C₁-C₁₈alkyl or unsubstituted or substituted aryl, or
R₁₃ and R₁₄, together with the nitrogen atom linking them, form an unsubstituted or substituted 5-, 6- or 7-membered ring which may contain further hetero atoms,
   with the proviso that
   at least one of the substituents R₁ to R₁₀ contains a quaternised nitrogen atom that is not bonded directly to one of the three rings A, B and/or C,
   as catalyst(s) for bleach reactions in cleaning formulations for hard surfaces.

Preferably the compounds of formula (1′) are used as catalysts for bleach reactions in dishwashing formulations. Such dishwashing formulations are preferably used in automatic dishwashing machines.

The preferences for all substituents of formula (1′) are the same as for those of the compound of formula (1), which are disclosed in page 2-page 8.

As examples of the radical R₅ in L′ of formula (2′) mention may be made especially —OH;

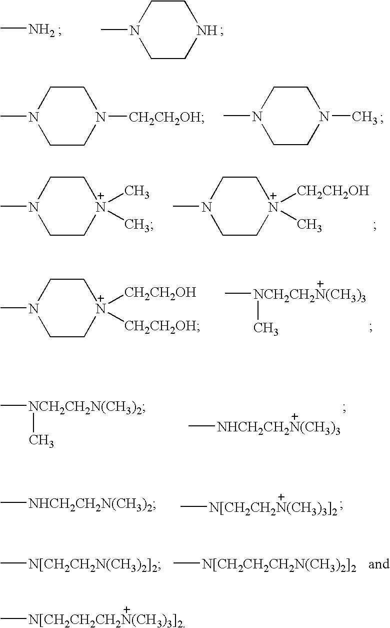

Of those, hydroxy is of special interest.

The preferred meanings given above for R₅ in L′ of formula (2′) apply also to R₁, R₂, R₃, R₄, R₅, R₇, R₈, R₉ and R₁₀ in L′ of formula (2′), but those radicals may additionally be hydrogen.

In accordance with one embodiment of the present invention, R₁, R₂, R₃, R₄, R₆, R₇, R₈, R₉ and R₁₀ in L′ of formula (2′) are hydrogen and R₅ in L′ of formula (2′) is a radical other than hydrogen having the definition and preferred meanings indicated above.

A preferred embodiment of the present invention relates to the use of at least one Mn(II)-complex of formula (3′c) and/or (3′d) 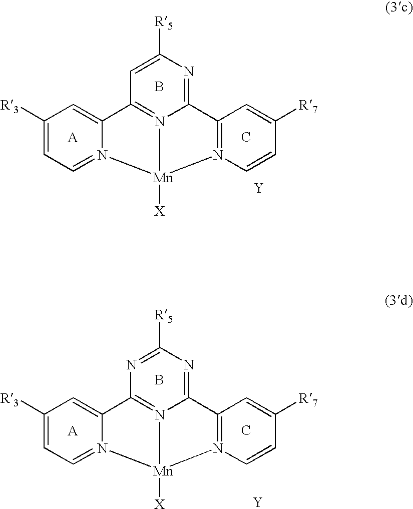
wherein
R₁₅ is 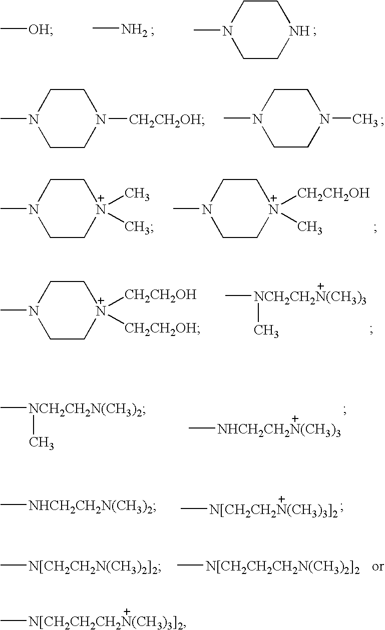
R′₃ and R′₇ are independently of each other are H; Cl; —OH; —NH₂; 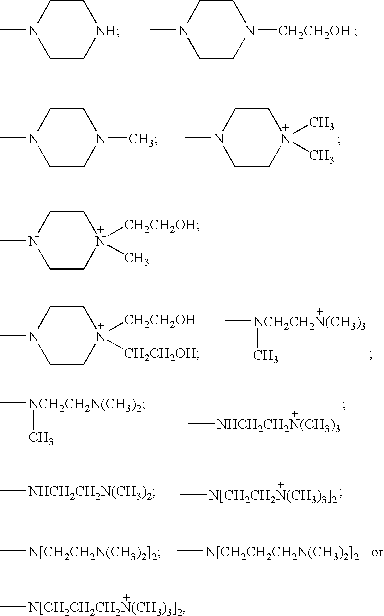
with the proviso that at least one of the substituents R′₃, R′₅ and R′₇ is 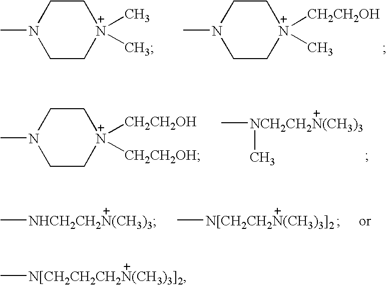
X is F⁻; Cl⁻; Br⁻; HOO⁻; CH₃COO⁻; HCOO⁻ or HO⁻, and
Y is CH₃COO⁻; HCOO⁻; ClO₄⁻; BF₄⁻; PF₆⁻; HSO₃⁻; HSO₄⁻; NO₃⁻; F⁻; Cl⁻; Br⁻ or I⁻.

A more preferred embodiment of the present invention relates to the use of at least one Mn(II)-complex of formula (3′c) and/or (3′d) 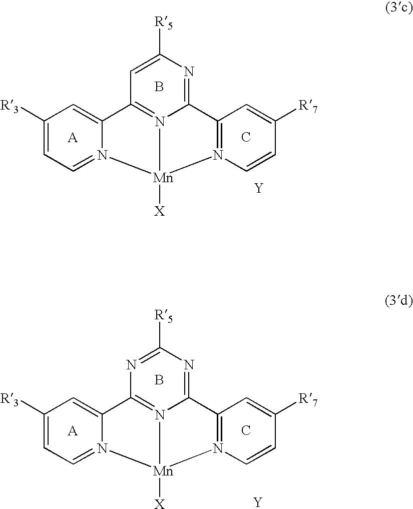
wherein
R′₅ is 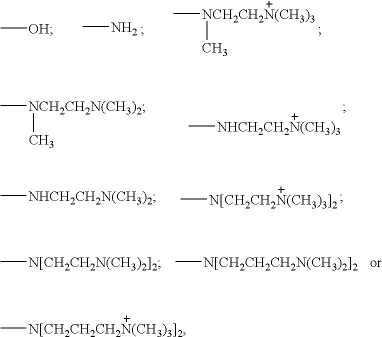
R′₃ is H; Cl; —OH; —NH₂; 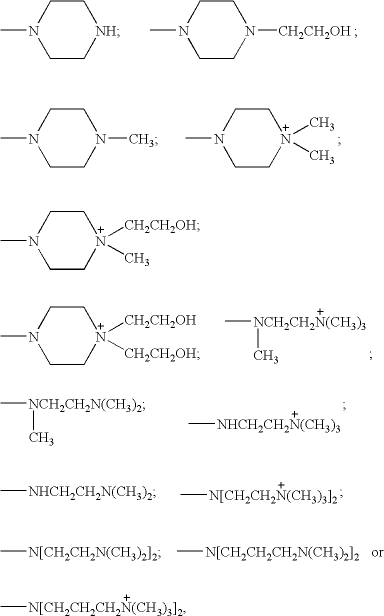
R′₇ is Cl; —OH; —NH₂; 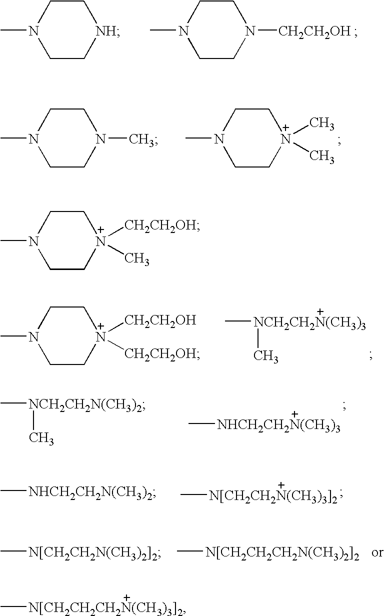
with the proviso that at least one of the substituents R′₃, and R′₇ is 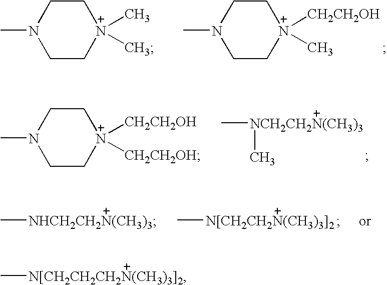
X is F⁻; Cl⁻; Br⁻; HOO⁻; CH₃COO⁻; HCOO⁻ or HO⁻, and
Y is CH₃COO⁻; HCOO⁻; ClO₄⁻; BF₄⁻; PF₆⁻; HSO₃⁻; HSO₄⁻; NO₃⁻; F⁻; Cl⁻; Br⁻ or I⁻.

Preferred as L′ are compounds of formulae (2′), (3′c) and (3′d) in which 1 quarternised nitrogen atom is present.

Also preferred as L′ are compounds of formulae (2′), (3′c) and (3′d) in which 2 or 3 quaternised nitrogen atoms are present.

Especially preferred as L′ are compounds of formulae (2′), (3′c) and (3′d) in which none of the quaternised nitrogen atoms is bonded directly to one of three rings A, B and/or C.

The metal complex compounds of formulae (1) and (1′) can be obtained analogously to known processes. They are obtained in a manner known per se by reacting at least one ligand L and/or L′ in the desired molar ratio with a metal compound, especially a metal salt, such as the chloride, to form the corresponding metal complex. The reaction is carried out, for example, in a solvent, such as water or a lower alcohol, such as ethanol, at a temperature of, for example, from 10 to 60° C., especially at room temperature.

Ligands L and L′ that are substituted by hydroxy can also be depicted in one or more tautomeric forms in accordance with the following scheme:

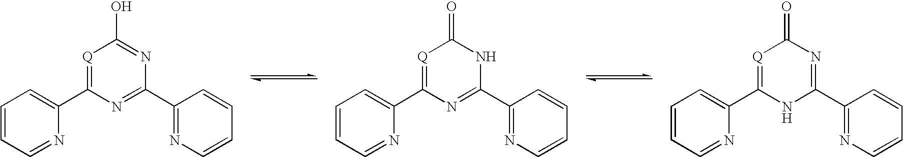

Compounds of the bispyridyl-pyrimidine type can also be prepared in a manner known per se [F. H. Case et al., J. Org. Chem. 1967, 32(5), 1591-1596]). For that purpose, for example, one part pyridine-2-carboxylate and one part ethyl acetate can be reacted with sodium hydride, and the intermediate obtained after aqueous working-up, a β-keto ester, reacted with 2-amidinopyridine, yielding the corresponding pyrimidine derivative which can be converted into the chlorine compounds by reaction with a chlorinating agent, such as, for example, PCl₅/POCl₃. Reaction of those compounds with amines, as desired in the presence of an excess of redox-active salts of transition metals, such as manganese, iron or ruthenium, in order to accelerate substitution, yields amine-substituted bispyridyl-pyrimidines. Preparation procedures using the latter two metal ions are described, for example, in J. Chem. Soc., Dalton Trans. 1990, 1405-1409 (E. C. Constable et al.) and New. J. Chem. 1992, 16, 855-867.

It has now been found that, in order to accelerate replacement of halide by amine on the bispyridyl-pyrimidine structure, it is also possible to use catalytic amounts of non-transition metal salts, such as, for example, zinc(II) salts, which substantially simplifies the reaction procedure and working-up.

Compounds of the bispyridyl-triazine type can be prepared analogously to known processes (e.g. Patent Applications EP 555 180 and EP 556 156 or F. H. Case et al., J. Am. Chem. Soc. 1959, 81, 905-906), by reacting two parts 2-cyanopyridine with urea or guanidine and a base.

The invention relates also to cleaning formulations for hard surfaces, especially cleaning formulations for table- and kitchen-ware and, among such formulations, preferably those for use in cleaning processes carried out by machine, which formulations comprise one of the above-described compounds of formula (1a) or (1′a) as bleach catalyst, and to a method of cleaning hard surfaces, especially table- and kitchen-ware, using such a bleach catalyst.

The present invention also relates to hard surface cleaning compositions (C1), preferably dishwashing compositions, comprising at least one compound of formula (1)
[LₙMeₘXₚ]^{z}Y_{q} (1),
wherein
Me is manganese, titanium, iron, cobalt, nickel or copper,
X is a coordinating or bridging radical,
n and m are each independently of the other an integer having a value of from 1 to 8,
p is an integer having a value of from 0 to 32,
z is the charge of the metal complex,
Y is a counter-ion,
q=z/(charge of Y), and
L is a ligand of formula (2) 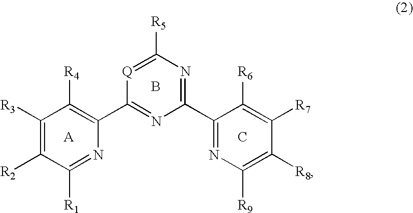
wherein
Q is N or CR₁₀,
R₁, R₂, R₃, R₄, R₅, R₆, R₇, R₈, R₉ and R₁₀ are each independently of the others hydrogen; unsubstituted or substituted C₁-C₁₈alkyl or unsubstituted or substituted aryl; cyano; halogen; nitro; —COOR₁₁ or —SO₃R₁₁ wherein
R₁₁ is in each case hydrogen, a cation or unsubstituted or substituted C₁-C₁₈alkyl or unsubstituted or substituted aryl;
   —SR₁₂, —SO₂R₁₂ or —OR₁₂ wherein
R₁₂ is in each case hydrogen or unsubstituted or substituted C₁-C₁₈alkyl or unsubstituted or substituted aryl;
   —NR₁₃R₁₄; —(C₁-C₁₈alkylene)-NR₁₃R₁₄; —N^{⊕}R₁₃R₁₄R₁₅; —(C₁-C₆alkylene)-N^{⊕}R₁₃R₁₄R₁₅; —N(R₁₂)—(C₁-C₆alkylene)-NR₁₃R₁₄; —N[(C₁-C₆alkylene)-NR₁₃R₁₄]₂; —N(R₁₂)—(C₁-C₆alkylene)-N^{⊕}R₁₃R₁₄R₁₅; —N[(C₁-C₆alkylene)-N^{⊕}R₁₃R₁₄R₁₅]₂; —N(R₁₂)—N—R₁₃R₁₄ or —N(R₁₂)—N^{⊕}R₁₃R₁₄R₁₅, wherein
R₁₂ is as defined above and
R₁₃, R₁₄ and R₁₅ are each independently of the other(s) hydrogen or unsubstituted or substituted C₁-C₁₈alkyl or unsubstituted or substituted aryl, or
R₁₃ and R₁₄, together with the nitrogen atom linking them, form an unsubstituted or substituted 5-, 6- or 7-membered ring which may contain further hetero atoms.

The above mentioned surface cleaning composition (C1) composition is preferably a dishwashing composition, more preferably an automatic dishwasher composition.

The preferences for all substituents of formula (1) are the same as disclosed on page 2-page 8.

A preferred embodiment relates to hard surface cleaning compositions (C2), preferably dishwashing compositions, comprising at least one Mn(II)-complex of formula (3c) and/or (3d) 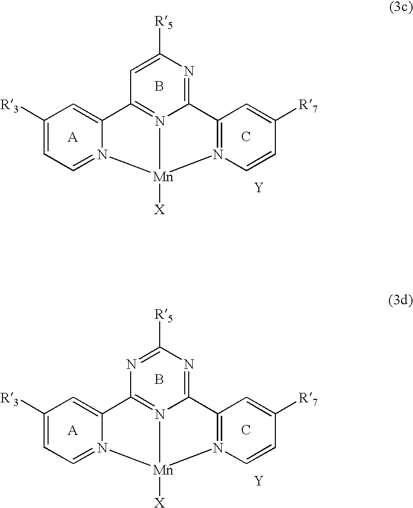
wherein
R′₅ is hydroxy; N-mono- or N,N-di-C₁-C₂alkylamino unsubstituted or substituted by hydroxy in the alkyl moiety; or —NR₁₃R₁₄; —(C₁-C₂alkylene)-NR₁₃R₁₄; —N(R₁₂)—(C₁-C₂alkylene)-NR₁₃R₁₄; —N[(C₁-C₂alkylene)-NR₁₃R₁₄]₂; or —N(R₁₂)—N—R₁₃R₁₄, wherein
   R₁₂ is hydrogen; C₁-C₄alkyl or unsubstituted phenyl or phenyl substituted by (substituted in the alkyl moiety by hydroxy) N-mono- or N,N-di-C₁-C₂alkylamino-, N-phenylamino-, N-naphthylamino-, phenyl-, phenoxy- or naphthyloxy, and
   R₁₃ and R₁₄ are each independently of the other hydrogen, unsubstituted or hydroxy-substituted C₁-C₄alkyl, unsubstituted phenyl or phenyl substituted as indicated above, or
   R₁₃ and R₁₄, together with the nitrogen atom linking them, form a pyrrolidine, piperidine, piperazine, morpholine or azepane ring that is unsubstituted or substituted by at least one unsubstituted C₁-C₄alkyl and/or substituted C₁-C₄alkyl, especially a pyrrolidine, piperidine, piperazine, morpholine or azepane ring, and
R′₃ and R′₇ are each independently of the other hydrogen; halogen; hydroxy; N-mono- or N,N-di-C₁-C₂alkylamino substituted by hydroxy in the alkyl moiety; or —NR₁₃R₁₄; —(C₁-C₂alkylene)-NR₁₃R₁₄; —N(R₁₂)—(C₁-C₂alkylene)-NR₁₃R₁₄; —N[(C₁-C₂alkylene)-NR₁₃R₁₄]₂; or —N(R₁₂)—N—R₁₃R₁₄, wherein
   R₁₂ is hydrogen; C₁-C₄alkyl or unsubstituted phenyl or phenyl substituted by (substituted in the alkyl moiety by hydroxy) N-mono- or N,N-di-C₁-C₂alkylamino-, N-phenylamino-, N-naphthylamino-, phenyl-, phenoxy- or naphthyloxy, and
   R₁₃ and R₁₄ are each independently of the other hydrogen; unsubstituted or hydroxy-substituted C₁-C₄alkyl, unsubstituted phenyl or phenyl substituted as indicated above, or
   R₁₃ and R₁₄, together with the nitrogen atom linking them, form a pyrrolidine, piperidine, piperazine, morpholine or azepane ring that is unsubstituted or substituted by at least one unsubstituted C₁-C₄alkyl and/or substituted C₁-C₄alkyl, especially a pyrrolidine, piperidine, piperazine, morpholine or azepane ring,
      X is F⁻; Cl⁻; Br⁻; HOO⁻; ⁻CH₃COO⁻; HCOO⁻ or HO⁻, and
      Y is CH₃COO⁻; HCOO⁻; ClO₄⁻; BF₄⁻; PF₆⁻; HSO₃⁻; HSO₄⁻; NO₃⁻; F⁻; Cl⁻; Br⁻ or I⁻.

The above mentioned surface cleaning composition (C2) composition is preferably a dishwashing composition, more preferably an automatic dishwasher composition.

A more preferred embodiment relates to hard surface cleaning compositions (C3), preferably dishwashing compositions, comprising at least one Mn(II)-complex of formula (3c) and/or (3d) 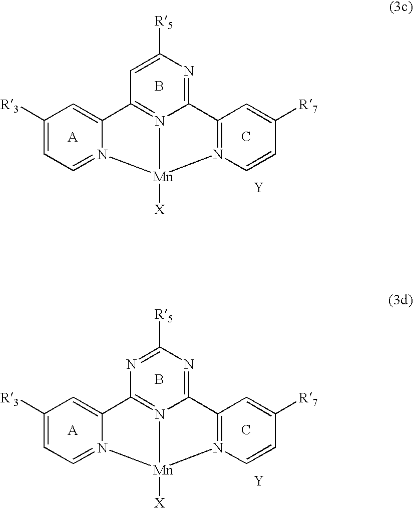
wherein
R′₅ is hydroxy; N-mono- or N,N-di-C₁-C₂alkylamino unsubstituted or substituted by hydroxy in the alkyl moiety; or —NH₂
R′₃ and R′₇ are each independently of the other hydrogen; Cl; hydroxy; N-mono- or N,N-di-C₁-C₂alkylamino substituted by hydroxy in the alkyl moiety; 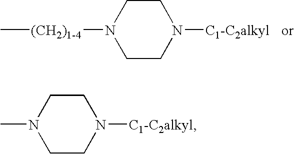
X is F⁻; Cl⁻; Br⁻; HOO⁻; CH₃COO⁻; HCOO⁻ or HO⁻, and
Y is CH₃COO⁻; HCOO⁻; ClO₄⁻; BF₄⁻; PF₆⁻; HSO₃⁻; HSO₄⁻; NO₃⁻; F⁻; Cl⁻; Br⁻ or I⁻.

The above mentioned surface cleaning composition (C3) composition is preferably a dishwashing composition, more preferably an automatic dishwasher composition.

The present invention relates to hardsurface cleaning compositions (C4), preferably dishwashing compositions, comprising at least one metal complex compounds of formula (1′)
[L′ₙMeₘXₚ]^{z}Y_{q} (1′),
wherein
Me is manganese, titanium, iron, cobalt, nickel or copper,
X is a coordinating or bridging radical,
n and m are each independently of the other an integer having a value of from 1 to 8,
p is an integer having a value of from 0 to 32,
z is the charge of the metal complex,
Y is a counter-ion,
q=z/(charge of Y), and
L′ is a ligand of formula (2′) 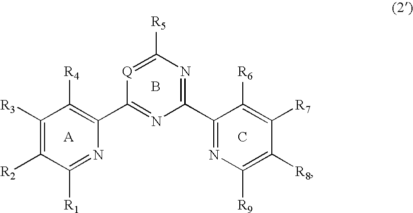
wherein
Q is N or CR₁₀,
R₁, R₂, R₃, R₄, R₅, R₆, R₇, R₈, R₉ and R₁₀ are each independently of the others hydrogen; unsubstituted or substituted C₁-C₁₈alkyl or unsubstituted or substituted aryl; cyano; halogen; nitro; —COOR₁₁ or —SO₃R₁₁ wherein
R₁₁ is in each case hydrogen, a cation or unsubstituted or substituted C₁-C₁₈alkyl or unsubstituted or substituted aryl;
   —SR₁₂, —SO₂R₁₂ or —OR₁₂ wherein
R₁₂ is in each case hydrogen or unsubstituted or substituted C₁-C₁₈alkyl or substituted or unsubstituted aryl;
   —NR₁₃R₁₄; —(C₁-C₆alkylene)-NR₁₃R₁₄; —N^{⊕}R₁₃R₁₄R₁₅; —(C₁-C₆alkylene)-N^{⊕}R₁₃R₁₄R₁₅; —N(R₁₂)—(C₁-C₆alkylene)-NR₁₃R₁₄; —N[(C₁-C₆alkylene)-NR₁₃R₁₄]₂; —N(R₁₂)—(C₁-C₆alkylene)-N^{⊕}R₁₃R₁₄R₁₅; —N[(C₁-C₆alkylene)-N^{⊕}R₁₃R₁₄R₁₅]₂; —N(R₁₂)—N—R₁₃R₁₄ or —N(R₁₂)—N^{⊕}R₁₃R₁₄R₁₅, wherein
R₁₂ is as defined above and
R₁₃, R₁₄ and R₁₅ are each independently of the other(s) hydrogen or unsubstituted or substituted C₁-C₁₈alkyl or substituted or unsubstituted aryl, or
R₁₃ and R₁₄, together with the nitrogen atom linking them, form an unsubstituted or substituted 5-, 6- or 7-membered ring which may contain further hetero atoms, with the proviso that,
   at least one of the substituents R₁ to R₁₀ contains a quaternised nitrogen atom that is not bonded directly to one of the three rings A, B and/or C.

The above mentioned surface cleaning composition (C4) composition is preferably a dishwashing composition, more preferably an automatic dishwasher composition.

The preferences for all substituents of formula (1′) are the same as disclosed on page 2-page 8.

A preferred embodiment relates to hard surface cleaning compositions (C5), preferably dishwashing compositions, more preferably automatic dishwashing compositions, comprising at least one Mn(II)-complex of formula (3′c) and/or (3′d) 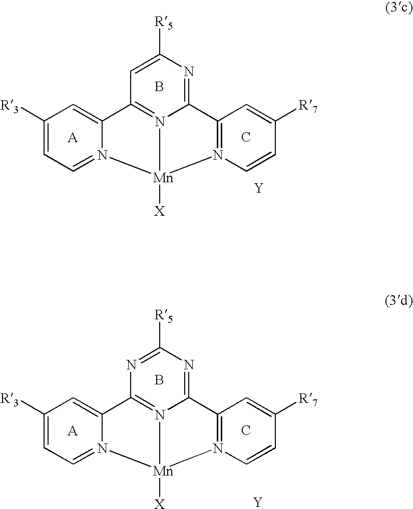
wherein
R′₅ is 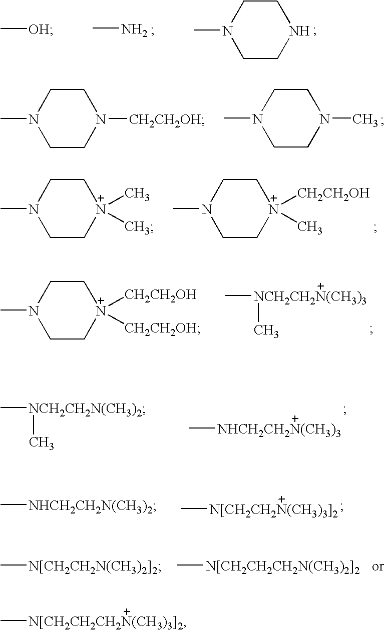
R′₃ and R′₇ are independently of each other are H; Cl; —OH; —NH₂; 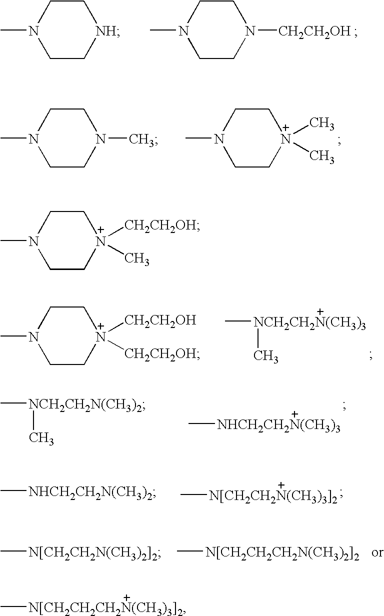
with the proviso that at least one of the substituents R′₃, R′₅ and R′₇ is 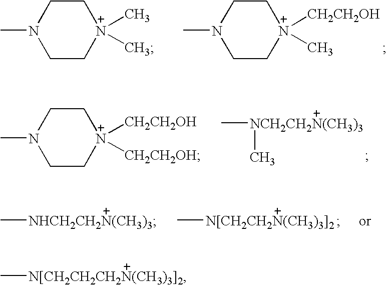
X is F⁻; Cl⁻; Br⁻; HOO⁻; CH₃COO⁻; HCOO⁻ or HO⁻, and
Y is CH₃COO⁻; HCOO⁻; ClO₄⁻; BF₄⁻; PF₆⁻; HSO₃⁻; HSO₄⁻; NO₃⁻; F⁻; Cl⁻; Br⁻ or I⁻.

The above mentioned surface cleaning composition (C5) composition is preferably a dishwashing composition, more preferably an automatic dishwasher composition.

A more preferred embodiment relates to hard surface cleaning compositions (C6), preferably dishwashing compositions, comprising at least one Mn(II)-complex of formula (3′c) and/or (3′d) 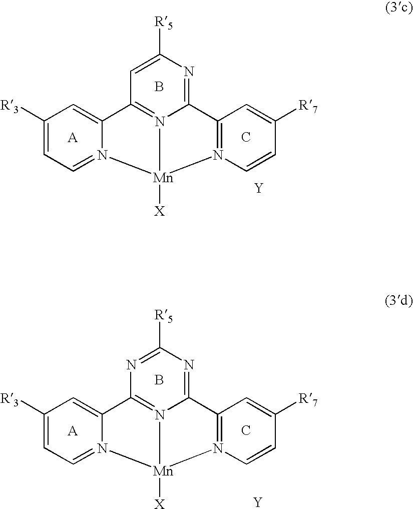
wherein
R′₅ is 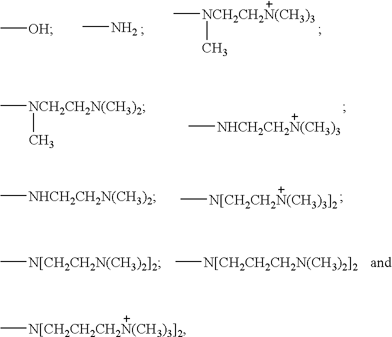
R′₃ is H; Cl; —OH; —NH₂; 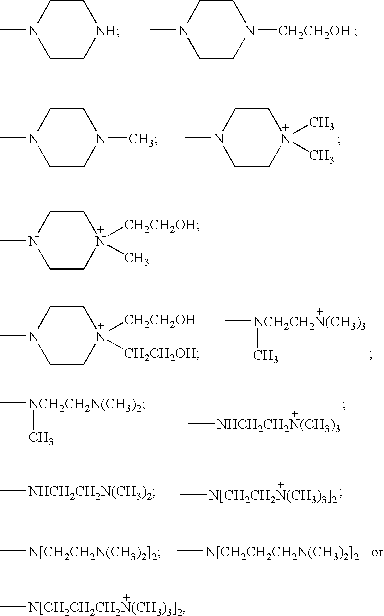
R′₇ is Cl; —OH; —NH₂; 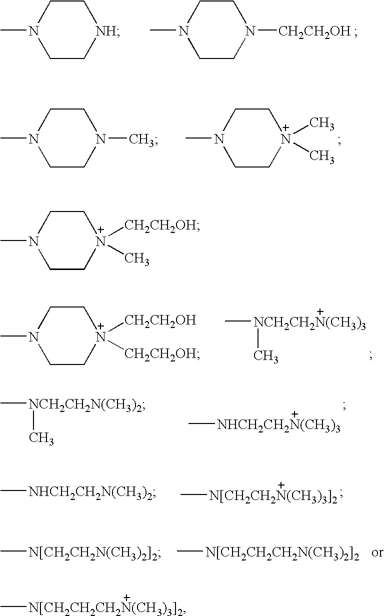
with the proviso that at least one of the substituents R′₃, and R′₇ is 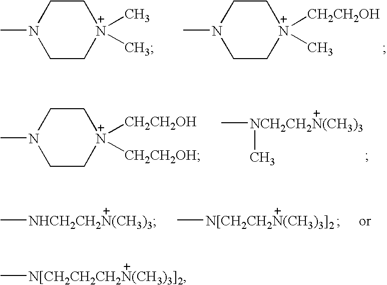
X is F⁻; Cl⁻; Br⁻; HOO⁻; CH₃COO⁻; HCOO⁻ or HO⁻, and
Y is CH₃COO⁻; HCOO⁻; ClO₄⁻; BF₄⁻; PF₆⁻; HSO₃⁻; HSO₄⁻; NO₃⁻; F⁻; Cl⁻; Br⁻ or I⁻.

The above mentioned surface cleaning composition (C6) composition is preferably a dishwashing composition, more preferably an automatic dishwasher composition.

Preferred Mn(II)-complexes in the hard surface cleaning composition (C4), (C5) and (C6) have just 1 quaternised nitrogen atom.

Also preferred Mn(II)-complexes in the hard surface cleaning composition (C4), (C5) and (C6) have 2 or 3 quaternised nitrogen atoms.

Especially preferred Mn(II)-complexes in the hard surface cleaning composition (C4), (C5) and (C6) are those in which none of the quaternised nitrogen atoms is bonded directly to one of the rings A, B or C.

The use according to the invention lies essentially in providing, in the presence of a hard surface soiled with coloured stains, conditions under which a peroxide-containing oxidising agent and the bleach catalyst according to formulae (1) and/or (1′) are able to react with one another with the aim of obtaining resulting products having a more strongly oxidising action. This can be brought about by separately adding the peroxy compound and the bleach catalyst to a formulation, which may optionally contain cleaning formulation. The method according to the invention is, however, advantageously carried out using a cleaning formulation for hard surfaces according to the invention that comprises the bleach catalyst and a peroxy-containing oxidising agent. The peroxy compound can also be added to the formulation separately, as such or in the form of a preferably aqueous formulation or suspension, when a peroxide-free cleaning formulation is used.

Depending upon the intended use, the conditions can be widely varied. For example, in addition to purely aqueous solutions, mixtures of water and suitable organic solvents also come into consideration as reaction medium. The amounts of peroxy compounds used are generally so selected that from 10 ppm (wherein ppm denoted parts per million by weight) to 10% active oxygen, preferably from 50 ppm to 5000 ppm active oxygen, are present in the solutions. The amount of bleach catalyst used also depends upon the intended use.

Depending upon the desired degree of activation, from 0.00001 mol to 0.025 mol, preferably from 0.0001 mol to 0.1 mol, of catalyst is used per mole of peroxy compound, but in special cases amounts above or below those limits may also be used.

The hard surface cleaning compositions, preferably dishwashing compositions herein can have any desired physical form; when in granular form, it is typical to limit water content, for example to less than about 10%, preferably less than about 7% free water, for best storage stability.

Unless otherwise indicated, the hard surface cleaning compositions of the invention may for example, be formulated as granular or power-form all-purpose; liquid, gel or paste-form; hand dishwashing agents or light duty dishwashing agents, especially those of the high-foaming type; machine dishwashing agents, including the various tabletted, granular, liquid and rinse-aid types for household and institutional use; liquid cleaning and disinfecting agents, including antibacterial hand-wash types, bathroom cleaners; as well as cleaning auxiliaries such as bleach additives and “stain-stick” or pre-treat types.

The invention relates also to a cleaning formulation for hard surface, preferably to a dishwashing formulation, more preferably to an automatic dishwashing formulation (especially for table- and kitchen-ware), that comprises from 0.001% by weight (wt-%) to 1 wt-%, especially from 0.005 wt-% to 0.1 wt-%, bleach catalyst according to formulae (1) and/or (1′) in addition to customary ingredients compatible with the bleach catalyst. The weight percentages are based on the total weight of the cleaning formulation. The bleach catalyst may, in a manner known in principle, be adsorbed on carriers and/or embedded in encapsulating substances.

The cleaning formulations according to the invention, which can be in the form of powder- or tablet-form solids, or homogeneous solutions or suspensions, may in principle comprise, in addition to the bleach catalyst used according to the invention, any known ingredients customary in such formulations. The formulations according to the invention may comprise especially builder substances, surface-active surfactants, peroxy compounds, water-miscible organic solvents, enzymes, sequestering agents, electrolytes, pH regulators and further adjuvants, such as silver-corrosion inhibitors, foam regulators, additional peroxy activators and also colourings and perfumes.

A cleaning formulation for hard surfaces according to the invention can also comprise abrasive constituents, especially from the group comprising quartz powders, wood flours, ground plastics, chalks and glass microbeads, and mixtures thereof. Abrasive substances are present in the cleaning formulations according to the invention preferably in amounts not exceeding 20 wt-%, especially in amounts of from 5 wt-% to 15 wt-%.

Amounts of the essential ingredients can vary within wide ranges, however preferred hard surface cleaning compositions, preferably dishwashing detergent compositions, more preferably automatic dishwashing detergent compositions, herein (which typically have a 1% aqueous solution pH of above 8, more preferably from 9.0 to 12, most preferably from 9.5 to 10.5) are those wherein there is present:
from 5 wt-% to 90 wt-%, preferably from 5 wt-% to 75 wt-%, of at least one builder;
from 0.1 wt-% to 40 wt-%, preferably from 0.5 wt-% to 30 wt-%, of at least one oxygen-based bleaching agent;
from 0.1 wt-% to 15 wt-%, preferably from 0.2 wt-% to 10 wt-%, of the surfactant system;
from 0.0001 wt-% to 1 wt-%, preferably from 0.001 wt-% to 0.05 wt-%, of at least one metal-containing bleach catalyst as described above; and
from 0.1 wt-% to 40 wt-%, preferably from 0.1 wt-% to 20 wt-% of an alkaline carrier.

All wt-%'s relate to the total weight of the surface cleaning compositions, preferably dishwashing detergent compositions, and more preferably automatic dishwashing detergent compositions.

Such fully-formulated embodiments typically further comprise from 0.1 wt-% to 15 wt-% of a polymeric dispersant, from 0.01 wt-% to 10 wt-% of a chelant, and from 0.00001 wt-% to 10 wt-% of a detersive enzyme, though further additional or adjunct ingredients may be present. Preferred hard surface cleaning compositions, preferably dishwashing detergent compositions, more preferably automatic dishwashing detergent compositions herein in granular form typically limit water content, for example to less than 7 wt-% free water, for best storage stability. All wt-%'s relate to the total weight of the surface cleaning compositions.

Water-soluble builder components that come into consideration in such low-alkalinity cleaning formulations include, in principle, any builders customarily used in machine dishwashing formulations, for example polymeric alkali metal phosphates, which may be in the form of their alkaline, neutral or acidic sodium or potassium salts. Examples thereof are tetrasodium diphosphate, disodium dihydrogen diphosphate, pentasodium triphosphate, sodium tripolyphosphate, so-called sodium hexametaphosphate and the corresponding potassium salts as well as mixtures of sodium and potassium salts. They may be present in amounts in the range of up to about 35 wt-%, based on the total formulation. Other possible builder components include the various water-soluble, alkali metal, ammonium or substituted ammonium borates, hydroxysulfonates and polyacetates. Preferred are the alkali metals, especially sodium, salts of such materials. Further possible water-soluble builder components are, for example, organic polymers of natural or synthetic origin, especially polycarboxylates, which, particularly in hard water areas, act as co-builders. There come into consideration, for example, polyacrylic acids and copolymers of maleic anhydride and acrylic acid and also the sodium salts of those polymeric acids. Commercially available products are, for example, Sokalan® CP 5 and PA 30 from BASF. Polymers of natural origin that can be used as co-builders include, for example, oxidised starch, as known, for example, from International Patent Application WO 94/05762, and polyamino acids, such as polyglutamic acid or polyaspartic acid. Further possible builder components are naturally occurring hydroxycarboxylic acids, e.g. mono- and di-hydroxysuccinic acid, α-hydroxypropionic acid and gluconic acid. There also come into consideration as builder components the salts of citric acid, especially sodium citrate. As sodium citrate there come into consideration anhydrous trisodium citrate and especially trisodium citrate dihydrate. Trisodium citrate dihydrate can be used in the form of a fine- or coarse-crystalline powder. Depending upon the pH value ultimately established in the formulations according to the invention, the acids corresponding to the mentioned co-builder salts may also be present. Alternate water-soluble, non-phosphorus organic builders can be used for their sequestering properties. Examples of polyacetate and polycarboxylate builders are the sodium, potassium, lithium, ammonium and substituted ammonium salts of ethylenediamine tetraacetic acid; nitrilotriacetic acid, tartrate monosuccinic acid, tartrate disuccinic acid, oxydisuccinic acid, carboxymethyloxysuccinic acid, mellitic acid, and sodium benzene polycarboxylate salts. Oxygen-based bleaching agents that come into consideration include alkali metal perborate, commercially available, e.g., in the form of mono- or tetra-hydrate; urea peroxyhydrate, alkali metal percarbonate, and sodium peroxide. Hydrogen peroxide sources are described in detail in the herein incorporated Kirk Othmer's Encyclopedia of Chemical Technology, 4^{th} Ed (1992, John Wiley & Sons), Vol. 4, pp. 271-300 “Bleaching Gents (Survey)”. Particularly preferred are sodium perborate tetrahydrate, sodium perborate monohydrate and sodium percarbonate. Percarbonate is especially preferred because of environmental issues associated with boron.

Highly preferred percarbonate can be in uncoated or coated form. The average particle size of uncoated percarbonate ranges from about 400 to about 1200 microns, most preferably from about 400 to about 600 microns.

The use of sodium percarbonate has advantages especially in cleaning formulations for table- and kitchen-ware, since it has a particularly advantageous effect on corrosion behaviour in glasses.

In addition, or especially alternatively, it is also possible for known peroxycarboxylic acids to be present, for example dodecane-diperacid or phthalimidoperoxycarboxylic acids, which may be unsubstituted or substituted on the aromatic moiety. Furthermore, the addition of small amounts of known bleaching agent stabilisers, for example phosphonates, borates or metaborates and metasilicates, and also magnesium salts, such as magnesium sulfate, may be advantageous.

As surfactant system, at least one surfactant selected from the list including anionic, nonionic, cationic, amphoteric, and zwitterionic surfactants is used. Such surfactants are well known in the detergent arts and are described at length in “Surface Active Agents and Detergents”, Vol. 2 by Schwartz, Perry and Birch, Interscience Publishers, Inc.; 1959, herein incorporated by reference. Low foaming surfactants are most suitable for machine dishwashing applications.

Preferred surfactants are one or a mixture of:

Anionic Surfactants

Anionic synthetic detergents can be broadly described as surface active compounds with one or more negatively charged functional groups. An important class of anionic compounds are the water-soluble salts, particularly the alkali metal salts, of organic sulfur reaction products having in their molecular structure an alkyl radical containing from about 6 to 24 carbon atoms and a radical selected from the group consisting of sulfonic and sulfuric acid ester radicals.

Primary Alkyl Sulfates
R_{α}OSO₃M,
wherein
R_{α} is a primary alkyl group of 8 to 18 carbon atoms and M is a solubilizing cation.

The alkyl group R_{α} may have a mixture of chain lengths. It is preferred that at least two-thirds of the R_{α} alkyl groups have a chain length of 8 to 14 carbon atoms. This will be the case if R_{α} is coconut alkyl, for example. The solubilizing cation may be a range of cations which are in general monovalent and confer water solubility. An alkali metal, notably sodium, is especially envisaged. Other possibilities are ammonium and substituted ammonium ions, such as trialkanolammonium or trialkylammonium.

Alkyl Ether Sulfates
R_{α}O(CH₂CH₂O)ₙSO₃M,
wherein
R_{α} is a primary alkyl group of 8 to 18 carbon atoms,
n has an average value in the range from 1 to 6 and
M is a solubilizing cation.

The alkyl group R_{α} may have a mixture of chain lengths. It is preferred that at least two-thirds of the R_{α} alkyl groups have a chain length of 8 to 14 carbon atoms. This will be the case if R_{α} is coconut alkyl, for example. Preferably n has an average value of 2 to 5.

Fatty Acid Ester Sulfonates
R_{β}CH(SO₃M)CO₂R_{χ},
wherein
R_{β} is an alkyl group of 6 to 16 atoms,
R_{χ} is an alkyl group of 1 to 4 carbon atoms and
M is a solubilizing cation.

The group R_{β} may have a mixture of chain lengths. Preferably at least two-thirds of these groups have 6 to 12 carbon atoms. This will be the case when the moiety R_{β}CH(−)CO₂(−) is derived from a coconut source, for instance. It is preferred that R_{χ} is a straight chain alkyl, notably methyl or ethyl.

Alkyl Benzene Sulfonates
R_{δ}ArSO₃M,
wherein
R_{δ} is an alkyl group of 8 to 18 carbon atoms,
Ar is a benzene ring (C₆H₄) and
M is a solubilizing cation.

The group R_{δ} may be a mixture of chain lengths. Straight chains of 11 to 14 carbon atoms are preferred.

Paraffin sulfonates having 8 to 22 carbon atoms, preferably 12 to 16 carbon atoms, in the alkyl moiety. These surfactants are commercially available as Hostapur® SAS from Clariant.

Olefin sulfonates having 8 to 22 carbon atoms, preferably 12 to 16 carbon atoms. U.S. Pat. No. 3,332,880 contains a description of suitable olefin sulfonates.

Organic phosphate based anionic surfactants include organic phosphate esters such as complex mono- or diester phosphates of hydroxyl-terminated alkoxide condensates, or salts thereof, included in the organic phosphate esters are phosphate ester derivatives of polyoxyalkylated alkylaryl phosphate esters, of ethoxylated linear alcohols and ethoxylates of phenol. Also included are nonionic alkoxylates having a sodium alkylenecarboxylate moiety linked to a terminal hydroxyl group of the nonionic through an ether bond. Counterions to the salts of all the foregoing may be those of alkali metal, alkaline earth metal, ammonium, alkanolammonium and alkylammonium types.

Particularly preferred anionic surfactants are the fatty acid ester sulfonates with formula:
R_{β}CH(SO₃M)CO₂R_{χ},
wherein
the moiety R_{β}CH(−)CO₂(−) is derived from a coconut source and
R_{χ} is either methyl or ethyl; primary alkyl sulfates with the formula:
R_{α}OSO₃M, wherein
R_{α} is a primary alkyl group of 10 to 18 carbon atoms and
M is a sodium cation; and paraffin sulfonates, preferably with 12 to 16 carbon atoms to the alkyl moiety.
Nonionic Surfactants

Nonionic surfactants can be broadly defined as surface active compounds with one or more uncharged hydrophilic substituents. A major class of nonionic surfactants consists of those compounds produced by the condensation of alkylene oxide groups with an organic hydrophobic material which may be aliphatic or alkyl aromatic in nature. The length of the hydrophilic or polyoxyalkylene radical which is condensed with any particular hydrophobic group can be readily adjusted to yield a water-soluble compound having the desired degree of balance between hydrophilic and hydrophobic elements. Illustrative, but not limiting examples, of various suitable nonionic surfactant types are:

Polyoxyalkene condensates of aliphatic carboxylic acids, whether linear- or branched-chain and unsaturated or saturated, especially ethoxylated and/or propoxylated aliphatic acids containing from about 8 to about 18 carbon atoms in the aliphatic chain and incorporating from about 2 to about 50 ethylene oxide and/or propylene oxide units. Suitable carboxylic acids include “coconut” fatty acids (derived from coconut oil) which contain an average of about 12 carbon atoms, “tallow” fatty acids (derived from tallow-class fats) which contain an average of about 18 carbon atoms, palmitic acid, myristic acid, stearic acid and lauric acid.

Polyoxyalkene condensates of aliphatic alcohols, whether linear- or branched-chain and unsaturated or saturated, especially ethoxylated and/or propoxylated aliphatic alcohols containing from about 6 to about 24 carbon atoms and incorporating from about 2 to about 50 ethylene oxide and/or propylene oxide units. Suitable alcohols include “coconut” fatty alcohol, “tallow” fatty alcohol, lauryl alcohol, myristyl alcohol and oleyl alcohol.

Ethoxylated fatty alcohols may be used alone or in admixture with anionic surfactants, especially the preferred surfactants above. The average chain lengths of the alkyl group R_{ε} in the general formula:
R_{ε}O(CH₂CH₂)ₙH
is from 6 to 20 carbon atoms. Notably the group R_{ε} may have chain lengths in a range from 9 to 18 carbon atoms.

The average value of n should be at least 2. The numbers of ethylene oxide residues may be a statistical distribution around the average value. However, as is known, the distribution can be affected by the manufacturing process or altered by fractionation after ethoxylation. Particularly preferred ethoxylated fatty alcohols have a group R_{ε} which has 9 to 18 carbon atoms while n is from 2 to 8.

Also included within this category are nonionic surfactants having a formula: 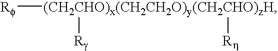
wherein
R_{φ} is a linear alkyl hydrocarbon radical having an average of 6 to 18 carbon atoms,
R_{γ} and R_{η} are each linear alkyl hydrocarbons of about 1 to about 4 carbon atoms,
x is an integer of from 1 to 6,
y is an integer of from 4 to 20 and
z is an integer from 4 to 25.

One preferred nonionic surfactant of the above formula is Poly-Tergent SLF-18, a registered trademark of the Olin Corporation, New Haven, Conn., having a composition of the above formula where R_{φ} is a C₆-C₁₀ linear alkyl mixture, R_{γ} and R_{η} are methyl, x averages 3, y averages 12 and z averages 16.

Another preferred nonionic surfactant is: 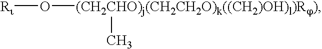
wherein
R_{ι} is a linear, aliphatic hydrocarbon radical having from about 4 to about 18 carbon atoms including mixtures thereof; and
R_{φ} is a linear, aliphatic hydrocarbon radical having from about 2 to about 26 carbon atoms including mixtures thereof;
j is an integer having a value of from 1 to about 3;
k is an integer having a value from 5 to about 30; and
l is an integer having a value of from 1 to about 3.

Most preferred are compositions in which j is 1, k is from about 10 to about 20 and l is 1. These surfactants are described in WO 94/22800. Other preferred nonionic surfactants are linear fatty alcohol alkoxylates with a capped terminal group, as described in U.S. Pat. No. 4,340,766 to BASF. Particularly preferred is Plurafac LF403 ex. BASF.

Another nonionic surfactant included within this category are compounds of formula:
R_{κ}—(CH₂CH₂O)_{q}H,
wherein
R_{κ} is a C₆-C₂₄ linear or branched alkyl hydrocarbon radical and q is a number from 2 to 50; more preferably R_{κ} is a C₈-C₁₈ linear alkyl mixture and q is a number from 2 to 15.

Polyoxyethylene or polyoxypropylene condensates of alkyl phenols, whether linear- or branched-chain and unsaturated or saturated, containing from about 6 to 12 carbon atoms and incorporating from about 2 to about 25 moles of ethylene oxide and/or propylene oxide.

Polyoxyethylene derivatives of sorbitan mono-, di-, and tri-fatty acid esters wherein the fatty acid component has between 12 and 24 carbon atoms. The preferred polyoxyethylene derivatives are of sorbitan monolaurate, sorbitan trilaurate, sorbitan monopalmitate, sorbitan tripalmitate, sorbitan monostearate, sorbitan monoisostearate, sorbitan tristearate, sorbitan monooleate, and sorbitan trioleate. The polyoxyethylene chains may contain between about 4 and 30 ethylene oxide units, preferably about 10 to 20. The sorbitan ester derivatives contain 1, 2 or 3 polyoxyethylene chains dependent upon whether they are mono-, di- or tri-acid esters.

Polyoxyethylene-polyoxypropylene block copolymers having formula:
HO(CH₂CH₂O)ₐ(CH(CH₃)CH₂O)_{b}(CH₂CH₂O)_{c}H or
HO(CH(CH₃)CH₂O)_{d}(CH₂CH₂O)ₑ(CH(CH₃)CH₂O)_{f}H,
wherein
a, b, c, d, e and f are integers from 1 to 350 reflecting the respective polyethylene oxide and polypropylene oxide blocks of said polymer.

The polyoxyethylene component of the block polymer constitutes at least about 10% of the block polymer. The material preferably has a molecular weight of between about 1,000 and 15,000, more preferably from about 1,500 to about 6,000. These materials are well-known in the art. They are available under the trademark “Pluronic” and “Pluronic R”, a product of BASF Corporation.

Amine oxides having formula:
R_{λ}R_{μ}R_{ν}N═O,
wherein
R_{λ}, R_{μ}, and R_{ν} are saturated aliphatic radicals or substituted saturated aliphatic radicals. Preferable amine oxides are those wherein R_{λ} is an alkyl chain of about 10 to about 20 carbon atoms and R_{μ} and R_{ν} are methyl or ethyl groups or both R_{λ} and R_{μ} are alkyl chains of about 6 to about 14 carbon atoms and R_{ν} is a methyl or ethyl group.
Amphoteric Synthetic Detergents

Amphoteric synthetic detergents can be broadly described as derivatives of aliphatic tertiary amines, in which the aliphatic radical may be straight chain or branched and wherein one of the aliphatic substituents contain from about 8 to about 18 carbons and one contains an anionic water-solubilizing group, i.e., carboxy, sulpho, sulphato, phosphato or phosphono. Examples of compounds falling within this definition are sodium 3-dodecylamino propionate and sodium 2-dodecylamino propane sulfonate.

Zwitterionic Synthetic Detergents

Zwitterionic synthetic detergents can be broadly described as derivatives of aliphatic quaternary ammonium, phosphonium and sulphonium compounds in which the aliphatic radical may be straight chained or branched, and wherein one of the aliphatic substituents contains from about 8 to about 18 carbon atoms and one contains an anionic water-solubilizing group, e.g., carboxy, sulpho, sulphato, phosphato or phosphono. These compounds are frequently referred to as betaines. Besides alkyl betaines, alkyl amino and alkyl amido betaines are encompassed within this invention.

Alkyl Glycosides
RₒO(R_{π}O)ₙ(Z₁)ₚ,
wherein
Rₒ is a monovalent organic radical (e.g., a monovalent saturated aliphatic, unsaturated aliphatic or aromatic radical such as alkyl, hydroxyalkyl, alkenyl, hydroxyalkenyl, aryl, alkylaryl, hydroxyalkylaryl, arylalkyl, alkenylaryl, arylalkenyl, etc.) containing from about 6 to about 30 (preferably from about 8 to 18 and more preferably from about 9 to about 13) carbon atoms;
R_{π} is a divalent hydrocarbon radical containing from 2 to about 4 carbon atoms such as ethylene, propylene or butylene (most preferably the unit (R_{π}O)n represents repeating units of ethylene oxide, propylene oxide and/or random or block combinations thereof;
n is a number having an average value of from 0 to about 12;
Z₁ represents a moiety derived from a reducing saccharide containing 5 or 6 carbon atoms (most preferably a glucose unit); and
p is a number having an average value of from 0.5 to about 10 preferably from about 0.5 to about 5.

Examples of commercially available materials from Henkel Kommanditgesellschaft Aktien of Dusseldorf, Germany include APG 300, 325 and 350 with P, being C₉-C₁₁, n is 0 and p is 1.3, 1.6 and 1.8-2.2 respectively; APG 500 and 550 with Rₒ is C₁₂-C₁₃, n is 0 and p is 1.3 and 1.8-2.2, respectively; and APG 600 with Rₒ being C₁₂-C₁₄, n is 0 and p is 1.3.

While esters of glucose are contemplated especially, it is envisaged that corresponding materials based on other reducing sugars, such as galactose and mannose are also suitable.

Particularly preferred nonionic surfactants are polyoxyethylene and polyoxypropylene condensates of linear aliphatic alcohols.

The preferred range of surfactant is from about 0.1 to 40 wt-%, more preferably from about 0.5 to 15 wt-% of the composition.

Non-ionic surfactants are preferred over anionic surfactants.

The surfactant system preferably comprises weakly foaming non-ionic surfactants, that serves for the better removal of fat-containing stains, as wetting agents and optionally within the framework of the preparation of the cleaning formulations as granulation auxiliaries. They may be present in an amount of up to 10 wt-%, based on the total weight of the hard surface cleaning composition, especially up to 5 wt-%, and preferably in the range of from 0.25 wt-% to 4 wt-%. Especially for use in machine dishwashing procedures it is customary to use extremely low-foam compounds. These include especially C₁₂-C₂₀alkylpolyethylene glycol-polypropylene glycol ethers each having up to 15 mol of ethylene oxide and propylene oxide units in the molecule. A particularly preferred non-ionic surfactant is derived from a straight chain fatty alcohol containing from about 16 to about 20 carbon atoms (C₁₆-C₂₀ alcohol), preferably a C₁₈alcohol, condensed with an average of from about 6 to about 15 moles, preferably from about 7 to about 12 moles, and most preferably from about 7 to about 9 moles of ethylene oxide per mole of alcohol. Preferably the ethoxylated nonionic surfactant so derived has a narrow ethoxylate distribution relative to the average. Also possible are blends thereof with more sophisticated surfactants, such as the polyoxypropylene/polyoxyethylene/poly-oxypropylene reverse block polymers. The PO/EO/PO polymer-type surfactants are well-known to have foam suppressing or defoaming action, especially in relation to common food soil ingredients such as egg.

Highly preferred automatic dishwashing detergent formulations herein wherein the non-ionic surfactant is present make use of ethoxylated monohydroxy alcohol or alkyl phenol and additionally comprise a polyoxyethylene, polyoxypropylene block polymeric compound; the ethoxylated monohydroxy alcohol or alkyl phenol fraction of the non-ionic surfactant comprising from about 20 wt-% to about 80 wt-%, preferably from about 30 wt-% to about 70 wt-%, based on the total weight of the non-ionic surfactant. Suitable block polyoxyethylene-polyoxypropylene polymeric compounds that meet the requirements described hereinbefore include those based on ethylene glycol, propylene glycol, glycerol, trimethylolpropane and ethylenediamine as initiator reactive hydrogen compound. Polymeric compounds made from a sequential ethoxylation and propoxylation of initiator compounds with a single reactive hydrogen atom, such as C₁₂-C₁₈ aliphatic alcohols, do not generally provide satisfactory suds control in the instant automatic dishwashing detergent formulations.

It is also possible, however, to use other known low-foam non-ionic surfactants, for example C₁₂-C₁₈alkylpolyethylene glycol-polybutylene glycol ethers each having up to 8 mol of ethylene oxide and butylene oxide units in the molecule, end-group-terminated alkylpolyalkylene glycol mixed ethers, and also the foaming but ecologically attractive C₈-C₁₄alkylpolyglucosides having a degree of polymerisation of approximately from 1 to 4 and/or C₁₂-C₁₄alkylpolyethylene glycols having from 3 to 8 ethylene oxide units in the molecule. Also suitable are surfactants from the glucamide family, e.g. alkyl-N-methyl-glucamides in which the alkyl moiety preferably consists of a fatty alcohol having a carbon chain length of C₆-C₁₄. In some cases it is advantageous for the described surfactants to be used in the form of mixtures, for example a combination of alkylpolyglycoside with fatty alcohol ethoxylates or of glucamide with alkylpolyglycosides. Preferred compositions of the present invention can optionally comprise limited quantities (up to about 2%) of nitrogen-containing nonionic surfactants, such as alkyldimethyl amineoxides or fatty glucosamides; when present, such surfactants normally require suds suppression e.g., by silicone suds suppressors.

In addition to the bleach catalysts according to formulae (1) and/or (1′) it is also possible to use further known transition metal salts or complexes known as bleach-activating active ingredients and/or conventional bleach activators, that is to say compounds that, under perhydrolysis conditions, yield unsubstituted or substituted perbenzo- and/or peroxo-carboxylic acids having from 1 to 10 carbon atoms, especially from 2 to 4 carbon atoms. Suitable bleach activators include the customary bleach activators, mentioned at the beginning, that carry O- and/or N-acyl groups having the indicated number of carbon atoms and/or unsubstituted or substituted benzoyl groups. Preference is given to polyacylated alkylenediamines, especially tetraacetylethylenediamine (TAED), acylated glycolurils, especially tetraacetylglycoluril (TAGU), N,N-diacetyl-N,N-dimethylurea (DDU), acylated triazine derivatives, especially 1,5-diacetyl-2,4-dioxohexahydro-1,3,5-triazine (DADHT), acylated phenylsulfonates, especially nonanoyloxybenzenesulfonate (NOBS) or isononanoyloxybenzenesulfonate, acylated polyvalent alcohols, especially triacetin, ethylene glycol diacetate and 2,5-di-acetoxy-2,5-dihydrofuran, and also acetylated sorbitol and mannitol and acylated sugar derivatives, especially pentaacetylglucose (PAG), sucrose polyacetate (SUPA), pentaacetylfructose, tetraacetylxylose and octaacetyllactose as well as acetylated, optionally N-alkylated glucamine and gluconolactone. It is also possible to use the combinations of conventional bleach activators known from German Patent Application DE-A-4443177.

The cleaning formulations according to the invention may also contain organic peroxides, especially diacyl peroxides. Nonlimiting examples of diacyl peroxides useful herein include dibenzoyl peroxide, lauroyl peroxide, and dicumyl peroxide.

The low-alkalinity machine dishwashing formulations according to the invention preferably comprise the customary alkaline carriers, for example alkali silicates, alkali carbonates and/or alkali hydrogen carbonates. The alkaline carrier are selected so that when the detergent is dissolved in water at a concentration of 1-10 g/L, the pH remains in the range of above about 8, preferably from about 9.0 to about 11. The alkaline carriers customarily used include alkali carbonates, hydrogen carbonates and alkali silicates having a molar ratio SiO₂/M₂O (M=alkali metal atom) of from 1:1 to 2.5:1, sodium borate, sodium hydroxide or mixtures thereof. The amount of the alkaline carrier in the instant automatic dishwashing detergent compositions is preferably from about 1 wt-% to about 50 wt-%, based on the total weight of the formulation. In a preferred embodiment, the alkaline carrier is present in the automatic dishwashing detergent formulation in an amount from about 5 wt-% to about 40 wt-%, preferably from about 10 wt-% to about 30 wt-%, based on the total weight of the formulation. An alkaline carrier system preferably used in the formulations according to the invention is a mixture of carbonate and hydrogen carbonate, preferably sodium carbonate and hydrogen carbonate, which mixture is present in an amount of up to 60 wt-%, preferably from 10 wt-% to 40 wt-%, based on the total weight of the formulation. Depending upon the pH value ultimately desired, the ratio of carbonate used to hydrogen carbonate used will vary, but usually an excess of sodium hydrogen carbonate is used, so that the ratio by weight of hydrogen carbonate to carbonate is generally from 1:1 to 15:1.

Alkali silicates can be present in amounts of up to 30 wt-%, based on the total weight of the formulation. It is preferable to dispense altogether with the use of the highly alkaline metasilicates as alkaline carriers. When present, sodium and potassium, especially sodium, silicates are preferred. Particularly preferred alkali metal silicates are granular hydrous sodium silicates having SiO₂:Na₂O ratio of about 2.0 or about 2.4. Most preferred is a granular hydrous sodium silicate having a SiO₂:Na₂O ratio of 2.0.

In a further preferred embodiment, the hard surface cleaning compositions according to the invention contain from 20 wt-% to 40 wt-% water-soluble organic builder, especially alkali citrate, from 5 wt-% to 15 wt-% alkali carbonate and from 20 wt-% to 40 wt-% alkali silicate based on the total weight of the formulation.

The present hard surface cleaning compositions, preferably dishwashing detergent formulations, more preferably automatic dishwashing detergent formulations, may contain one or more corrosion inhibitors or anti-tarnish aids. Such materials are preferred components of machine dishwashing compositions especially in certain European countries where the use of electroplated nickel silver and sterling silver is still comparatively common in domestic flatware, or when aluminium protection is a concern and the composition is low in silicate. When present, such protecting materials are preferably incorporated at low levels, e.g., from about 0.01 wt-% to about 5 wt-%, based on the total weight of the formulation, preferably dishwashing detergent formulation, more preferably automatic dishwashing detergent formulation. Suitable corrosion inhibitors include paraffin oil, unsubstituted or substituted benzotriazole and comparable compounds; mercaptans or thiols including thionaphtol and thioanthranol; divalent phenols, trivalent phenols, Aluminium fatty acid salts, such as aluminium tristearate, and manganese, titanium, zirconium, hafnium, vanadium, cobalt or cerium salts and/or complexes, in which the said metals are present in one of oxidation states II, III, IV, V and VI. The formulator will recognize that such materials will generally be used judiciously and in limited quantities so as to avoid any tendency to produce spots or films on glassware or to compromise the bleaching action of the compositions. Suitable Corrosion inhibitors are disclosed in U.S. Pat. No. 5,480,576, GB2297096, EP636688, GB2283494 and EP 690122.

In addition, the formulations according to the invention may comprise enzymes, such as proteases, amylases, pullulanases, cutinases and lipases, for example proteases such as BLAP®, Optimase®, Opticlean®, Maxacal®, Maxapem®, Esperase® and/or Savinase®, amylases such as Termamyl®, Amylase-LT®, Maxamyl® and/or Duramyl®, lipases such as Lipolase®, Lipomax®, Lumafast® and/or Lipozym®. The enzymes which may be used can, as described e.g. in International Patent Applications WO 92/11347 and WO 94/23005, be adsorbed on carriers and/or embedded in encapsulating substances in order to safeguard them against premature inactivation. They are present in the cleaning formulations according to the invention preferably in amounts not exceeding 5 wt-%, especially in amounts of from 0.1 wt-% to 1.2 wt-%, based on the total weight of the formulation.

Amylases: The present invention preferably makes use of amylases having improved stability in detergents, especially improved oxidative stability. Such amylases are non-limitingly illustrated by the following: (a) An amylase according to WO 94/02597, Novo Nordisk A/S, published Feb. 3, 1994, as further illustrated by a mutant in which substitution is made, using alanine or threonine (preferably threonine), of the methionine residue located in position 197 of the B. licheniformis alpha-amylase, known as TERMAMYL®, or the homologous position variation of a similar parent amylase, such as B. amyloliquefaciens, B. subtilis, or B. stearothermophilus; (b) Stability-enhanced amylases as described by Genencor International in a paper entitled “Oxidatively Resistant alpha-Amylases” presented at the 207th American Chemical Society National Meeting, Mar. 13-17, 1994, by C. Mitchinson. Therein it was noted that bleaches in automatic dishwashing detergents inactivate alpha-amylases but that improved oxidative stability amylases have been made by Genencor from B. licheniformis NCIB8061. Any other oxidative stability-enhanced amylase can be used.

Proteases: Protease enzymes are usually present in preferred embodiments of the invention at levels between 0.001 wt-% and 5 wt-%, based on the total weight of the formulation. The proteolytic enzyme can be of animal, vegetable or microorganism (preferred) origin. More preferred is serine proteolytic enzyme of bacterial origin. Purified or nonpurified forms of enzyme may be used. Proteolytic enzymes produced by chemically or genetically modified mutants are included by definition, as are close structural enzyme variants. Suitable commercial proteolytic enzymes include Alcalase®, Esperase®, Durazyme®, Savinase®, Maxatase®, Maxacal®, and Maxapem® 15 (protein engineered Maxacal). Purafect® and subtilising BPN and BPN′ are also commercially available.

When present, lipases comprise from about 0.001 wt-% to about 0.01 wt-%, based on the total weight of the formulation and are optionally combined with from about 1 wt-% to about 5 wt-% of a surfactant having limesoap-dispersing properties, such as an alkyldimethylamine N-oxide or a sulfobetaine. Suitable lipases for use herein include those of bacterial, animal and fungal origin, including those from chemically or genetically modified mutants. When incorporating lipases into the instant compositions, their stability and effectiveness may in certain instances be enhanced by combining them with small amounts (e.g., less than 0.5 wt-% of the composition) of oily but non-hydrolyzing materials.

The enzyme-containing hard surface cleaning compositions, preferably dishwashing detergent formulations, more preferably automatic dishwashing detergent formulations herein may optionally also comprise from about 0.001% to about 10%, preferably from about 0.005% to about 8%, most preferably from about 0.01% to about 6%, by weight of an enzyme stabilizing system. The enzyme stabilizing system can be any stabilizing system which is compatible with the detersive enzyme. Such a system may be inherently provided by other formulation actives, or be added separately, e.g., by the formulator or by a manufacturer of detergent-ready enzymes. Such stabilizing systems can, for example, comprise calcium ion, boric acid, propylene glycol, short chain carboxylic acids, boronic acids, and mixtures thereof, and are designed to address different stabilization problems depending on the type and physical form of the detergent composition.

One stabilizing approach is the use of water-soluble sources of calcium and/or magnesium ions in the finished compositions which provide such ions to the enzymes. Calcium ions are generally more effective than magnesium ions and are preferred herein if only one type of cation is being used. Typical detergent compositions, especially liquids, will comprise from about 1 to about 30, preferably from about 2 to about 20, more preferably from about 8 to about 12 millimoles of calcium ion per litre of finished detergent composition, though variation is possible depending on factors including the multiplicity, type and levels of enzymes incorporated. Preferably water-soluble calcium or magnesium salts are employed, including for example calcium chloride, calcium hydroxide, calcium formate, calcium malate, calcium maleate, calcium hydroxide and calcium acetate; more generally, calcium sulfate or magnesium salts corresponding to the exemplified calcium salts may be used. Further increased levels of Calcium and/or Magnesium may of course be useful, for example for promoting the grease-cutting action of certain types of surfactant.

Another stabilizing approach is by use of borate species. See Severson, U.S. Pat. No. 4,537,706. Borate stabilizers, when used, may be at levels of up to 10 wt-% or more of the composition though more typically, levels of up to about 3 wt-% of boric acid or other borate compounds such as borax or orthoborate are suitable for liquid detergent use. Substituted boric acids such as phenylboronic acid, butaneboronic acid, p-bromophenylboronic acid or the like can be used in place of boric acid and reduced levels of total boron in detergent compositions may be possible though the use of such substituted boron derivatives. Stabilizing systems of certain hard surface cleaning compositions, for example automatic dishwashing compositions, may further comprise from 0 to about 10 wt-%, preferably from about 0.01 wt-% to about 6 wt-%, of chlorine bleach scavengers, added to prevent chlorine bleach species present in many water supplies from attacking and inactivating the enzymes, especially under alkaline conditions. While chlorine levels in water may be small, typically in the range from about 0.5 ppm to about 1.75 ppm, the available chlorine in the total volume of water that comes in contact with the enzyme, for example during dish- or fabric-washing, can be relatively large; accordingly, enzyme stability to chlorine in-use is sometimes problematic. Since perborate or percarbonate, which have the ability to react with chlorine bleach, may present in certain of the instant compositions in amounts accounted for separately from the stabilizing system, the use of additional stabilizers against chlorine, may, most generally, not be essential, though improved results may be obtainable from their use. Suitable chlorine scavenger anions are widely known and readily available, and, if used, can be salts containing ammonium cations with sulfite, bisulfite, thiosulfite, thiosulfate, iodide, etc. Antioxidants such as carbamate, ascorbate, etc., organic amines such as ethylenediamine-tetracetic acid (EDTA) or alkali metal salt thereof, monoethanolamine (MEA), and mixtures thereof can likewise be used. Likewise, special enzyme inhibition systems can be incorporated such that different enzymes have maximum compatibility. Other conventional scavengers such as bisulfate, nitrate, chloride, sources of hydrogen peroxide such as sodium perborate tetrahydrate, sodium perborate monohydrate and sodium percarbonate, as well as phosphate, condensed phosphate, acetate, benzoate, citrate, formate, lactate, malate, tartrate, salicylate, etc., and mixtures thereof can be used if desired. In general, since the chlorine scavenger function can be performed by ingredients separately listed under better recognized functions, (e.g., hydrogen peroxide sources), there is no absolute requirement to add a separate chlorine scavenger unless a compound performing that function to the desired extent is absent from an enzyme-containing embodiment of the invention; even then, the scavenger is added only for optimum results. Moreover, the formulator will exercise a chemist's normal skill in avoiding the use of any enzyme scavenger or stabilizer which is majorly incompatible, as formulated, with other reactive ingredients. In relation to the use of ammonium salts, such salts can be simply admixed with the detergent composition but are prone to adsorb water and/or liberate ammonia during storage. Accordingly, such materials, if present, are desirably protected in a particle such as that described in U.S. Pat. No. 4,652,392.

Peroxidase enzymes are optionally useful in the present invention. They are used for “solution bleaching,” i.e. to prevent transfer of dyes or pigments removed from substrates during wash operations to other substrates in the wash solution. Peroxidase enzymes are known in the art, and include, for example, horseradish peroxidase, ligninase, and haloperoxidase such as chloro- and bromo-peroxidase.

The t hard surface cleaning compositions, preferably dishwashing detergent formulation, more preferably automatic dishwashing detergent formulation of the invention can optionally contain foam suppressors, for example alkyl phosphate ester suds suppressor, silicone suds suppressor, or combinations thereof. Levels in general are from 0 wt-% to about 10 wt-%, preferably, from about 0.001 wt-% to about 5 wt-%, based on the total weight of the formulation. Typical levels tend to be low, e.g., from about 0.01 wt-% to about 3 wt-% when a silicone suds suppressor is used. Preferred non-phosphate compositions omit the phosphate ester component entirely.

Highly preferred silicone suds suppressors are the compounded types known for use in laundry detergents such as heavy-duty granules, although types hitherto used only in heavy-duty liquid detergents may also be incorporated in the instant compositions. For example, polydimethylsiloxanes having trimethylsilyl or alternate endblocking units may be used as the silicone. These may be compounded with silica and/or with surface-active nonsilicon components, as illustrated by a suds suppressor comprising 12 wt-% silicone/silica, 18 wt-% stearyl alcohol and 70 wt-% starch in granular form.

Preferred alkyl phosphate esters contain from 16-20 carbon atoms. Highly preferred alkyl phosphate esters are monostearyl acid phosphate or monooleyl acid phosphate, or salts thereof, particularly alkali metal salts, or mixtures thereof.

It has been found preferable to avoid the use of simple calcium-precipitating soaps as antifoams in the present compositions as they tend to deposit on the dishware. Indeed, phosphate esters are not entirely free of such problems and the formulator will generally choose to minimize the content of potentially depositing antifoams in the instant compositions.

Other examples for foam suppressors are paraffin, paraffin/alcohol combinations, or bisfatty acid amides.

The hard surface cleaning compositions, preferably dishwashing detergent formulations, more preferably automatic dishwashing detergent formulations herein may also optionally contain one or more heavy metal chelating agents, such as hydroxyethyldiphosphonate, (HEDP). More generally, chelating agents suitable for use herein can be selected from the group consisting of amino carboxylates, amino phosphonates, polyfunctionally-substituted aromatic chelating agents and mixtures thereof. Other suitable chelating agents for use herein are the commercial DEQUEST series, and chelants from Nalco, Inc.

Aminocarboxylates useful as optional chelating agents include ethylenediaminetetracetates, N-hydroxyethylethylenediaminetriacetates, nitrilotriacetates, ethylenediamine tetraproprionates, triethylenetetraaminehexacetates, diethylenetriamine-pentaacetates, and ethanoldiglycines, alkali metal, ammonium, and substituted ammonium salts thereof and mixtures thereof.

Aminophosphonates are also suitable for use as chelating agents in the compositions of the invention when at least low levels of total phosphorus are permitted in detergent compositions, and include ethylenediaminetetrakis (methylenephosphonates). Preferably, these aminophosphonates do not contain alkyl or alkenyl groups with more than about 6 carbon atoms.

A highly preferred biodegradable chelator for use herein is ethylenediamine disuccinate (“EDDS”).

If utilized, these chelating agents or transition-metal selective sequestrants will generally comprise from about 0.001 wt-% to about 10 wt-%, more preferably from about 0.05 wt-% to about 1 wt-%, based on the total weight of the formulation, preferably dishwashing detergent formulations, more preferably automatic dishwashing detergent formulations herein.

Preferred hard surface cleaning compositions, preferably dishwashing detergent formulations, more preferably automatic dishwashing detergent formulations herein may additionally contain a dispersant polymer. When present, a dispersant polymer is typically at levels in the range from 0 wt-% to about 25 wt-%, preferably from about 0.5 wt-% to about 20 wt-%, more preferably from about 1 wt-% to about 8 wt-%, based on the total weight of the formulation. Dispersant polymers are useful for improved filming performance of the present dishwasher detergent compositions, especially in higher pH embodiments, such as those in which wash pH exceeds about 9.5. Particularly preferred are polymers, which inhibit the deposition of calcium carbonate or magnesium silicate on dishware.

Suitable polymers are preferably at least partially neutralized or alkali metal, ammonium or substituted ammonium (e.g., mono-, di- or triethanolammonium) salts of polycarboxylic acids. The alkali metal, especially sodium salts are most preferred. While the molecular weight of the polymer can vary over a wide range, it preferably is from about 1,000 to about 500,000, more preferably is from about 1,000 to about 250,000.

Unsaturated monomeric acids that can be polymerized to form suitable dispersant polymers include acrylic acid, maleic acid (or maleic anhydride), fumaric acid, itaconic acid, aconitic acid, mesaconic acid, citraconic acid and methylenemalonic acid. The presence of monomeric segments containing no carboxylate radicals such as methyl vinyl ether, styrene, ethylene, etc. is suitable provided that such segments do not constitute more than about 50 wt-% by weight of the dispersant polymer.

Copolymers of acrylamide and acrylate having a molecular weight of from about 3,000 to about 100,000, preferably from about 4,000 to about 20,000, and an acrylamide content of less than about 50 wt-%, preferably less than about 20 wt-%, by weight of the dispersant polymer can also be used. Most preferably, such dispersant polymer has a molecular weight of from about 4,000 to about 20,000 and an acrylamide content of from about 0 wt-% to about 15 wt-%, based on the total weight of the polymer.

Particularly preferred dispersant polymers are low molecular weight modified polyacrylate copolymers. Such copolymers contain as monomer units: a) from about 90 wt-% to about 10 wt-%, preferably from about 80 wt-% to about 20 wt-% acrylic acid or its salts and b) from about 10 wt-% to about 90 wt-%, preferably from about 20 wt-% to about 80 wt-% of a substituted acrylic monomer or its salt and have the general formula: —[(C(Rₐ)C(R_{b})(C(O)OR_{c})] wherein the apparently unfilled valencies are in fact occupied by hydrogen and at least one of the substituents Rₐ, R_{b}, or R_{c}, preferably Rₐ or R_{b}, is a 1 to 4 carbon alkyl or hydroxyalkyl group; Rₐ or R_{b} can be a hydrogen and R_{c} can be a hydrogen or alkali metal salt. Most preferred is a substituted acrylic monomer wherein Rₐ is methyl, R_{b} is hydrogen, and R_{c} is sodium.

Suitable low molecular weight polyacrylate dispersant polymer preferably has a molecular weight of less than about 15,000, preferably from about 500 to about 10,000, most preferably from about 1,000 to about 5,000. The most preferred polyacrylate copolymer for use herein has a molecular weight of about 3,500 and is the fully neutralized form of the polymer comprising about 70 wt-% acrylic acid and about 30 wt-% methacrylic acid.

Other dispersant polymers useful herein include the polyethylene glycols and polypropylene glycols having a molecular weight of from about 950 to about 30,000.

Yet other dispersant polymers useful herein include the cellulose sulfate esters such as cellulose acetate sulfate, cellulose sulfate, hydroxyethyl cellulose sulfate, methylcellulose sulfate, and hydroxypropylcellulose sulfate. Sodium cellulose sulfate is the most preferred polymer of this group.

Other suitable dispersant polymers are the carboxylated polysaccharides, particularly starches, celluloses and alginates.

Yet another group of acceptable dispersants are the organic dispersant polymers, such as polyaspartate.

Depending on whether a greater or lesser degree of compactness is required, filler materials can also be present in the instant hard surface cleaning compositions, preferably dishwashing detergent formulations, more preferably automatic dishwashing detergent formulations. These include sucrose, sucrose esters, sodium sulfate, potassium sulfate, etc., in amounts up to about 70 wt-%, preferably from 0 wt-% to about 40 wt-%, based on the total weight of the formulation, preferably dishwashing detergent formulations, more preferably automatic dishwashing detergent formulations. Preferred filler is sodium sulfate, especially in good grades having at most low levels of trace impurities.

Sodium sulfate used herein preferably has a purity sufficient to ensure it is non-reactive with bleach; it may also be treated with low levels of sequestrants, such as phosphonates or EDDS in magnesium-salt form. Note that preferences, in terms of purity sufficient to avoid decomposing bleach, applies also to pH-adjusting component ingredients, specifically including any silicates used herein.

Organic solvents that can be used in the cleaning formulations according to the invention, especially when the latter are in liquid or paste form, include alcohols having from 1 to 4 carbon atoms, especially methanol, ethanol, isopropanol and tert-butanol, diols having from 2 to 4 carbon atoms, especially ethylene glycol and propylene glycol, and mixtures thereof, and the ethers derivable from the mentioned classes of compound. Such water-miscible solvents are present in the cleaning formulations according to the invention preferably in amounts not exceeding 20 wt-%, especially in amounts of from 1 wt-% to 15 wt-%, based on the total weight of the formulation.

Many hard surface cleaning compositions, preferably dishwashing detergent formulations, more preferably automatic dishwashing detergent formulations herein will be buffered, i.e., they are relatively resistant to pH drop in the presence of acidic soils. However, other compositions herein may have exceptionally low buffering capacity, or may be substantially unbuffered. Techniques for controlling or varying pH at recommended usage levels more generally include the use of not only buffers, but also additional alkalis, acids, pH-jump systems, dual compartment containers, etc., and are well known to those skilled in the art. Certain hard surface cleaning compositions, preferably dishwashing detergent formulations, more preferably automatic dishwashing detergent formulations, comprise a pH-adjusting component selected from water-soluble alkaline inorganic salts and water-soluble organic or inorganic builders. The pH-adjusting components are selected so that when the hard surface cleaning compositions, preferably dishwashing detergent formulations, more preferably automatic dishwashing detergent formulations is dissolved in water at a concentration of 1,000-5,000 ppm, the pH remains in the range of above about 8, preferably from about 9.5 to about 11. The preferred nonphosphate pH-adjusting component can be selected from the group consisting of:
(i) sodium carbonate or sesquicarbonate;
(ii) sodium silicate, preferably hydrous sodium silicate having SiO₂:Na₂O ratio of from about 1:1 to about 2:1, and mixtures thereof with limited quantities of sodium metasilicate;
(iii) sodium citrate;
(iv) citric acid;
(v) sodium bicarbonate;
(vi) sodium borate, preferably borax;
(vii) sodium hydroxide; and
(viii) mixtures of (i)-(vii).

Preferred embodiments contain low levels of silicate (i.e. from about 3% to about 10% SiO₂). Illustrative of highly preferred pH-adjusting component systems of this specialized type are binary mixtures of granular sodium citrate with anhydrous sodium carbonate, and three-component mixtures of granular sodium citrate trihydrate, citric acid monohydrate and anhydrous sodium carbonate.

The amount of the pH adjusting component in compositions used for automatic dishwashing is preferably from about 1 wt-% to about 50 wt-%, based on the total weight of the formulation. In a preferred embodiment, the pH-adjusting component is present in the composition in an amount from about 5 wt-% to about 40 wt-%, preferably from about 10 wt-% to about 30 wt-%, based on the total weight of the formulation.

For compositions herein having a pH between about 9.5 and about 11 of the initial wash solution, particularly preferred automatic dishwashing detergent formulations embodiments comprise, by weight of the automatic dishwashing detergent formulations, from about 5 wt-% to about 40 wt-%, preferably from about 10 wt-% to about 30 wt-%, most preferably from about 15 wt-% to about 20 wt-%, of sodium citrate with from about 5 wt-% to about 30 wt-%, preferably from about 7 wt-% to 25 wt-%, most preferably from about 8 wt-% to about 20 wt-% sodium carbonate.

The essential pH-adjusting system can be complemented (i.e. for improved sequestration in hard water) by other optional detergency builder salts selected from nonphosphate detergency builders known in the art, which include the various water-soluble, alkali metal, ammonium or substituted ammonium borates, hydroxysulfonates, polyacetates, and polycarboxylates. Preferred are the alkali metal, especially sodium, salts of such materials. Alternate water-soluble, non-phosphorus organic builders can be used for their sequestering properties. Examples of polyacetate and polycarboxylate builders are the sodium, potassium, lithium, ammonium and substituted ammonium salts of ethylenediamine tetraacetic acid; nitrilotriacetic acid, tartrate monosuccinic acid, tartrate disuccinic acid, oxydisuccinic acid, carboxymethoxysuccinic acid, mellitic acid, and sodium benzene polycarboxylate salts. Automatic dishwashing detergent compositions may further comprise water-soluble silicates. Water-soluble silicates herein are any silicates which are soluble to the extent that they do not adversely affect spotting/filming characteristics of the automatic dishwashing detergent formulations composition.

Examples of silicates are sodium metasilicate and, more generally, the alkali metal silicates, particularly those having a SiO₂:Na₂O ratio in the range 1.6:1 to 3.2:1; and layered silicates, such as the layered sodium silicates described in U.S. Pat. No. 4,664,839, issued May 12, 1987 to H. P. Rieck. NaSKS-6® is a crystalline layered silicate marketed by Hoechst (commonly abbreviated herein as “SKS-6”). Unlike zeolite builders, Na SKS-6 and other water-soluble silicates useful herein do not contain aluminum. NaSKS-6 is the δ-Na₂SiO₅ form of layered silicate and can be prepared by methods such as those described in German DE-A-3,417,649 and DE-A-3,742,043. SKS-6 is a preferred layered silicate for use herein, but other such layered silicates, such as those having the general formula NaMSiₓO₂ₓ₊₁.yH2O wherein M is sodium or hydrogen, x is a number from 1.9 to 4, preferably 2, and y is a number from 0 to 20, preferably 0 can be used. Various other layered silicates from Hoechst include NaSKS-5, NaSKS-7 and NaSKS-11, as the α, β- and γ-forms. Other silicates may also be useful, such as for example magnesium silicate, which can serve as a crispening agent in granular formulations, as a stabilizing agent for oxygen bleaches, and as a component of suds control systems.

Silicates particularly useful in hard surface cleaning compositions, preferably dishwashing detergent formulations, more preferably automatic dishwashing detergent formulations applications include granular hydrous 2-ratio silicates such as BRITESIL® H20 from PQ Corp., and the commonly sourced BRITESIL® H24 though liquid grades of various silicates can be used when the automatic dishwasher composition has liquid form. Within safe limits, sodium metasilicate or sodium hydroxide alone or in combination with other silicates may be used in an automatic dishwasher context to boost wash pH to a desired level.

Hydrotrope materials such as sodium benzene sulfonate, sodium toluene sulfonate, sodium cumene sulfonate, etc., can be present, e.g., for better dispersing surfactant.

Bleach-stable perfumes (stable as to odor); and bleach-stable dyes can also be added to the present compositions in appropriate amounts. Other common detergent ingredients consistent with the spirit and scope of the present invention are not excluded.

Since hard surface cleaning compositions, preferably dishwashing detergent formulations, more preferably automatic dishwashing detergent formulations compositions herein can contain water-sensitive ingredients or ingredients which can co-react when brought together in an aqueous environment, it is desirable to keep the free moisture content of the hard surface cleaning compositions, preferably dishwashing detergent formulations, more preferably automatic dishwashing detergent formulations at a minimum, e.g., 7 wt-% or less, preferably 4 wt-% or less of the hard surface cleaning compositions, preferably dishwashing detergent formulations, more preferably automatic dishwashing detergent formulations; and to provide packaging which is substantially impermeable to water and carbon dioxide. Coating measures may be used to protect the ingredients from each other and from air and moisture.

The hard surface cleaning compositions, preferably dishwashing detergent formulations, more preferably automatic dishwashing detergent formulations according to the present invention may also comprise at least one organic peroxy acid.

As organic peroxy acid any known peroxy acid can be used. For example, mono- or poly-peroxy acids having at least 1 carbon atoms, preferably from 1 to 20 carbon atoms, in the alkyl chain. It is also possible to use the corresponding precursor of these acids.

Preferred are organic peroxy acids of formula 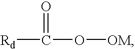
wherein
M signifies hydrogen or a cation,
R_{d} signifies unsubstituted C₁-C₁₈alkyl; substituted C₁-C₁₈alkyl; unsubstituted aryl; substituted aryl; —(C₁-C₆alkylene)-aryl, wherein the alkylene and/or the alkyl group may be substituted; and phthalimidoC₁-C₈alkylene, wherein the phthalimido and/or the alkylene group may be substituted.

The C₁-C₁₈alkyl radicals mentioned are generally, for example, straight-chain or branched alkyl radicals, such as methyl, ethyl, n-propyl, isopropyl, n-butyl, sec-butyl, isobutyl, tert-butyl or straight-chain or branched pentyl, hexyl, heptyl or octyl.

Preference is given to C₁-C₁₂alkyl radicals, especially C₁-C₈alkyl radicals and preferably C₁-C₄alkyl radicals. The mentioned alkyl radicals may be unsubstituted or substituted e.g. by hydroxy, C₁-C₄alkoxy, sulfo or by sulfato.

The corresponding unsubstituted alkyl radicals are preferred. Very special preference is given to methyl and ethyl, especially methyl.

Examples of aryl radicals that generally come into consideration are phenyl or naphthyl each unsubstituted or substituted by C₁-C₄alkyl, C₁-C₄alkoxy, halogen, cyano, nitro, carboxy, sulfo, hydroxy, amino, N-mono- or N,N-di-C₁-C₄alkylamino unsubstituted or substituted by hydroxy in the alkyl moiety, N-phenylamino, N-naphthylamino, phenyl, phenoxy or by naphthyloxy. Preferred substituents are C₁-C₄alkyl, C₁-C₄alkoxy, phenyl and hydroxy.

The C₁-C₆alkylene groups mentioned are, for example, straight-chain or branched alkylene radicals, such as methylene, ethylene, n-propylene or n-butylene. C₁-C₄alkylene groups are preferred. The alkylene radicals mentioned may be unsubstituted or substituted, for example by hydroxy or C₁-C₄alkoxy.

The cation M can be any suitable cation or mixtures of cations. Examples of cations that generally come into consideration are alkali metal cations, such as lithium, potassium and especially sodium, alkaline earth metal cations, such as magnesium and calcium, and ammonium cations. The alkali metal cations, especially sodium, are preferred.

Very preferred organic peroxy acids and their salts are those of formula 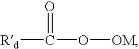
wherein
M signifies hydrogen or an alkali metal, and
R′^{d} signifies unsubstituted C₁-C₄alkyl; phenyl; —C₁-C₂alkylene-phenyl or phthalimidoC₁-C₈alkylene.

Especially preferred is CH₃COOOH and its alkali salts.

Especially preferred is also e-phthalimido peroxy hexanoic acid and its alkali salts.

Instead of the peroxy acid it is also possible to use organic peroxy acid precursors and H₂O₂. Such precursors are the corresponding carboxyacid or the corresponding carboxyanhydrid or the corresponding carbonylchlorid, or amides, or esters, which can form the peroxy acids on perhydrolysis. Such reactions are commonly known.

The preparation of the solid formulations according to the invention is problem-free and can be carried out in a manner known in principle, for example by spray-drying or granulation, peroxy compound and bleach catalyst optionally being added separately at a later stage.

Cleaning formulations according to the invention in the form of aqueous solutions or solutions containing some other customary solvent are produced especially advantageously by simply mixing together the ingredients, which can be introduced into an automatic mixer as such or in the form of a solution.

The formulation according to the invention may take the form of a complete dishwashing detergent or in the alternative may take the form of a separate bleaching additive. In the latter case the bleaching additive may used for removing coloured stains on crockery/kitchenware in a separate liquor before the items are washed in a dishwasher. The bleaching additive can also be used in a liquor together with either a bleach-free washing agent or a bleach-containing washing agent as a bleach booster.

The dishwasher detergent formulations can take a variety of physical forms such as, for example, powder, granule, tablets (tabs) and liquid.

When a liquid formulation is used, the formulation may comprise a thickener, such as is commonly used to increase the viscosity of the formulation and appeal to the consumer. Preferred examples of such thickeners include Xantham gum, cellulose derivatives and polyacrylic acid derivatives.

The formulations according to the invention are preferably in the form of powder-form, granular or tablet-form preparations which can be produced in a manner known per se, for example by mixing, granulating, roller-compacting and/or by spray-drying those components which are able to withstand thermal stress and then mixing in the more sensitive components, which include especially enzymes, bleaching agents and the bleach catalyst.

The advantages of tablets reside in the ease of dispensing and convenience in handling. Tablets are the most compact form of solid detergent formulation and usually have a volumetric density of, for example, from 0.9 to 1.3 kg/litre. To achieve rapid dissolution, such tablets may contain special dissolution aids:
carbonate/hydrogen carbonate/citric acid as effervescents;
disintegrators, such as cellulose, carboxymethyl cellulose or cross-linked poly(N-vinyl-pyrrolidone);
rapidly dissolving materials, such as sodium (potassium) acetates, or sodium (potassium) citrates;
rapidly dissolving, water-soluble, rigid coating agents, such as dicarboxylic acids.

The tablets may also comprise combinations of such dissolution aids.

A tablet may include a tabletting aid such as polyethyleneglycol.

For the preparation of the cleaning formulations according to the invention in tablet-form, the procedure is preferably as follows: all the constituents are mixed together in a mixer and the mixture is compressed by means of conventional tabletting presses, for example eccentric tablet presses or rotary tablet presses, at compression pressures in the range of from 200·10⁵ Pa to 1500·10⁵ Pa. There are thus obtained in a problem-free manner tablets that are resistant to breaking but nevertheless dissolve sufficiently rapidly under use conditions and have a flexural strength usually exceeding 150 N. A tablet so produced preferably has a weight of from 15 g to 40 g, especially from 20 g to 30 g, at a diameter of from 35 mm to 40 mm.

Tablets can also be prepared in the form of twin or multi layer tablets. This allows a separation of sensitive compounds from each other (e.g. bleach from enzymes, or persalts from catalysts) thereby stabilizing the formulation.

The preparation of the formulations according to the invention in the form of non-dusty, storage-stable pourable powders and/or granules having high bulk densities in the range of from 800 to 1000 g/l can be carried out by, in a first process step, mixing the builder components with at least some of the liquid mixing components, thus increasing the bulk density of the resulting premix, and subsequently—if desired after intermediate drying—combining the other constituents of the formulation, including the bleach catalyst, with the premix so obtained.

The invention relates also to granules that comprise the catalysts according to the invention and are suitable for incorporation into a powder-form, granular or tablet-form dishwashing detergent. Such granules preferably comprise:
a) from 1 to 99 wt-%, preferably from 1 to 40 wt-%, and especially from 1 to 30 wt-%, of at least one metal complex compound of formula (1) and/or (1′),
b) from 1 to 99 wt-%, preferably from 10 to 99 wt-%, and especially from 20 to 80 wt-%, of at least one binder,
c) from 0 to 20 wt-%, especially from 1 to 20 wt-%, of at least one encapsulating material,
d) from 0 to 20 wt-% of at least further additive and
e) from 0 to 20 wt-% of water.

As binder (b) there come into consideration water-soluble, dispersible or water-emulsifiable anionic dispersants, non-ionic dispersants, polymers and waxes.

The anionic dispersants used are, for example, commercially available water-soluble anionic dispersants for dyes, pigments etc.

The following products, especially, come into consideration: condensation products of aromatic sulfonic acids and formaldehyde, condensation products of aromatic sulfonic acids with unsubstituted or chlorinated diphenyls or diphenyl oxides and optionally formaldehyde, (mono-/di-)alkylnaphthalenesulfonates, sodium salts of polymerised organic sulfonic acids, sodium salts of polymerised alkylnaphthalenesulfonic acids, sodium salts of polymerised alkylbenzenesulfonic acids, alkylarylsulfonates, sodium salts of alkyl polyglycol ether sulfates, polyalkylated polynuclear arylsulfonates, methylene-linked condensation products of arylsulfonic acids and hydroxyarylsulfonic acids, sodium salts of dialkylsulfosuccinic acid, sodium salts of alkyl diglycol ether sulfates, sodium salts of polynaphthalenemethane-sulfonates, lignosulfonates or oxylignosulfonates and heterocyclic polysulfonic acids. Especially suitable anionic dispersants are condensation products of naphthalenesulfonic acids with formaldehyde, sodium salts of polymerised organic sulfonic acids, (mono-/di-)-alkylnaphthalenesulfonates, polyalkylated polynuclear arylsulfonates, sodium salts of polymerised alkylbenzenesulfonic acid, lignosulfonates, oxylignosulfonates and condensation products of naphthalenesulfonic acid with a polychloromethyldiphenyl.

Suitable non-ionic dispersants are especially compounds having a melting point of, preferably, at least 35° C. that are emulsifiable, dispersible or soluble in water, for example the following compounds:
1. fatty alcohols having from 8 to 22 carbon atoms, especially cetyl alcohol;
2. addition products of, preferably, from 2 to 80 mol of alkylene oxide, especially ethylene oxide, wherein some of the ethylene oxide units may have been replaced by substituted epoxides, such as styrene oxide and/or propylene oxide, with higher unsaturated or saturated monoalcohols, fatty acids, fatty amines or fatty amides having from 8 to 22 carbon atoms or with benzyl alcohols, phenyl phenols, benzyl phenols or alkyl phenols, the alkyl radicals of which have at least 4 carbon atoms;
3. alkylene oxide, especially propylene oxide, condensation products (block polymers);
4. ethylene oxide/propylene oxide adducts with diamines, especially ethylenediamine;
5. reaction products of a fatty acid having from 8 to 22 carbon atoms and a primary or secondary amine having at least one hydroxy-lower alkyl or lower alkoxy-lower alkyl group, or alkylene oxide addition products of such hydroxyalkyl-group-containing reaction products;
6. sorbitan esters, preferably having long-chain ester groups, or ethoxylated sorbitan esters, such as polyoxyethylene sorbitan monolaurate having from 4 to 10 ethylene oxide units or polyoxyethylene sorbitan trioleate having from 4 to 20 ethylene oxide units;
7. addition products of propylene oxide with a tri- to hexa-hydric aliphatic alcohol having from 3 to 6 carbon atoms, e.g. glycerol or pentaerythritol; and
8. fatty alcohol polyglycol mixed ethers, especially addition products of from 3 to 30 mol of ethylene oxide and from 3 to 30 mol of propylene oxide with aliphatic monoalcohols having from 8 to 22 carbon atoms.
   Especially suitable non-ionic dispersants are surfactants of formula
   R₂₃—O-(alkylene-O)ₙ—R₂₄ (9),
   wherein
R₂₃ is C₈-C₂₂alkyl or C₈-C₁₈alkenyl;
R₂₄ is hydrogen; C₁-C₄alkyl; a cycloaliphatic radical having at least 6 carbon atoms; or benzyl;
“alkylene” is an alkylene radical having from 2 to 4 carbon atoms and
n is a number from 1 to 60.

The substituents R₂₃ and R₂₄ in formula (9) are advantageously each the hydrocarbon radical of an unsaturated or, preferably, saturated aliphatic monoalcohol having from 8 to 22 carbon atoms. The hydrocarbon radical may be straight-chain or branched.

R₂₃ and R₂₄ are preferably each independently of the other an alkyl radical having from 9 to 14 carbon atoms.

Aliphatic saturated monoalcohols that may into consideration include natural alcohols, e.g. lauryl alcohol, myristyl alcohol, cetyl alcohol or stearyl alcohol, and also synthetic alcohols, e.g. 2-ethylhexanol, 1,1,3,3-tetramethylbutanol, octan-2-ol, isononyl alcohol, trimethylhexanol, trimethylnonyl alcohol, decanol, C₉-C₁₁oxo-alcohol, tridecyl alcohol, isotridecyl alcohol and linear primary alcohols (Alfols) having from 8 to 22 carbon atoms. Some examples of such Alfols are Alfol (8-10), Alfol (9-11), Alfol (10-14), Alfol (12-13) and Alfol (16-18). (“Alfol” is a registered trade mark of the company Sasol Limited).

Unsaturated aliphatic monoalcohols are, for example, dodecenyl alcohol, hexadecenyl alcohol and oleyl alcohol.

The alcohol radicals may be present singly or in the form of mixtures of two or more components, e.g. mixtures of alkyl and/or alkenyl groups that are derived from soybean fatty acids, palm kernel fatty acids or tallow oils.

(Alkylene-O) chains are preferably bivalent radicals of the formulae

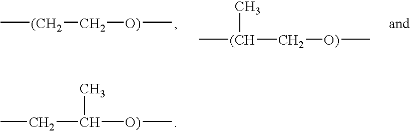

Examples of a cycloaliphatic radical include cycloheptyl, cyclooctyl and, preferably, cyclohexyl.

As non-ionic dispersants there come into consideration preferably surfactants of formula 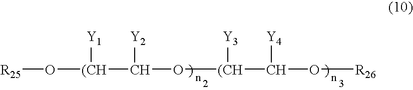
wherein
R₂₅ is C₈-C₂₂alkyl;
R₂₆ is hydrogen or C₁-C₄alkyl;
Y₁, Y₂, Y₃ and Y₄ are each independently of the others hydrogen, methyl or ethyl;
n₂ is a number from 0 to 8; and
n₃ is a number from 2 to 40.

Further important non-ionic dispersants correspond to formula 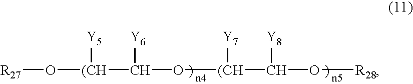
wherein
R₂₇ is C₉-C₁₄alkyl;
R₂₈ is C₁-C₄alkyl;
Y₅, Y₆, Y₇ and Y₈ are each independently of the others hydrogen, methyl or ethyl, one of the radicals Y₅, Y₆ and one of the radicals Y₇, Y₈ always being hydrogen; and
n₄ and n₅ are each independently of the other an integer from 4 to 8.

The non-ionic dispersants of formulae (9) to (11) can be used in the form of mixtures. For example, as surfactant mixtures there come into consideration non-end-group-terminated fatty alcohol ethoxylates of formula (9), e.g. compounds of formula (9) wherein

R₂₃ is C₈-C₂₂alkyl,

R₂₄ is hydrogen and

the alkylene-O chain is the radical —(CH₂—CH₂—O)—,

and also end-group-terminated fatty alcohol ethoxylates of formula (11).

Examples of non-ionic dispersants of formulae (9), (10) and (11) include reaction products of a C₁₀-C₁₃fatty alcohol, e.g. a C₁₃oxo-alcohol, with from 3 to 10 mol of ethylene oxide, propylene oxide and/or butylene oxide and the reaction product of one mol of a C₁₃fatty alcohol with 6 mol of ethylene oxide and 1 mol of butylene oxide, it being possible for the addition products each to be end-group-terminated with C₁-C₄alkyl, preferably methyl or butyl.

Such dispersants can be used singly or in the form of mixtures of two or more dispersants.

Instead of, or in addition to, the anionic or non-ionic dispersant, the granules according to the invention may comprise a water-soluble organic polymer as binder. Such polymers may be used singly or in the form of mixtures of two or more polymers.

Water-soluble polymers that come into consideration are, for example, polyethylene glycols, copolymers of ethylene oxide with propylene oxide, gelatin, polyacrylates, polymethacrylates, polyvinylpyrrolidones, vinylpyrrolidones, vinyl acetates, polyvinylimidazoles, polyvinylpyridine-N-oxides, copolymers of vinylpyrrolidone with long-chain x-olefins, copolymers of vinylpyrrolidone with vinylimidazole, poly(vinylpyrrolidone/dimethylaminoethyl methacrylates), copolymers of vinylpyrrolidone/dimethylaminopropyl methacrylamides, copolymers of vinylpyrrolidone/dimethylaminopropyl acrylamides, quaternised copolymers of vinylpyrrolidones and dimethylaminoethyl methacrylates, terpolymers of vinylcaprolactam/vinylpyrrolidone/dimethylaminoethyl methacrylates, copolymers of vinylpyrrolidone and methacrylamidopropyl-trimethylammonium chloride, terpolymers of caprolactam/vinylpyrrolidone/dimethylaminoethyl methacrylates, copolymers of styrene and acrylic acid, polycarboxylic acids, polyacrylamides, carboxymethyl cellulose, hydroxymethyl cellulose, polyvinyl alcohols, polyvinyl acetate, hydrolysed polyvinyl acetate, copolymers of ethyl acrylate with methacrylate and methacrylic acid, copolymers of maleic acid with unsaturated hydrocarbons, and also mixed polymerisation products of the mentioned polymers.

Of those organic polymers, special preference is given to polyethylene glycols, carboxymethyl cellulose, polyacrylamides, polyvinyl alcohols, polyvinylpyrrolidones, gelatin, hydrolysed polyvinyl acetates, copolymers of vinylpyrrolidone and vinyl acetate, and also polyacrylates, copolymers of ethyl acrylate with methacrylate and methacrylic acid, and polymethacrylates.

Suitable water-emulsifiable or water-dispersible binders also include paraffin waxes.

Encapsulating materials (c) include especially water-soluble and water-dispersible polymers and waxes. Of those materials, preference is given to polyethylene glycols, polyamides, polyacrylamides, polyvinyl alcohols, polyvinylpyrrolidones, gelatin, hydrolysed polyvinyl acetates, copolymers of vinylpyrrolidone and vinyl acetate, and also polyacrylates, paraffins, fatty acids, copolymers of ethyl acrylate with methacrylate and methacrylic acid, and polymethacrylates.

Further additives (d) that come into consideration are, for example, wetting agents, dust removers, water-insoluble or water-soluble dyes or pigments, and also dissolution accelerators, optical brighteners and sequestering agents.

The preparation of the granules according to the invention is carried out, for example, starting from:

a) a solution or suspension with a subsequent drying/shaping step or

b) a suspension of the active ingredient in a melt with subsequent shaping and solidification.

a) First of all the anionic or non-ionic dispersant and/or the polymer and, optionally, the further additives are dissolved in water and stirred, if desired with heating, until a homogeneous solution is obtained. The catalyst according to the invention is then dissolved or suspended in the resulting aqueous solution. The solids content of the solution should preferably be at least 30 wt-%, especially from 40 to 50 wt-%, based on the total weight of the solution. The viscosity of the solution is preferably less than 200 mPas.

The aqueous solution so prepared, comprising the catalyst according to the invention, is then subjected to a drying step in which all water, with the exception of a residual amount, is removed, solid particles (granules) being formed at the same time. Known methods are suitable for producing the granules from the aqueous solution. In principle, both continuous methods and discontinuous methods are suitable. Continuous methods are preferred, especially spray-drying and fluidised bed granulation processes.

Especially suitable are spray-drying processes in which the active ingredient solution is sprayed into a chamber with circulating hot air. The atomisation of the solution is effected e.g. using unitary or binary nozzles or is brought about by the spinning effect of a rapidly rotating disc. In order to increase the particle size, the spray-drying process may be combined with an additional agglomeration of the liquid particles with solid nuclei in a fluidised bed that forms an integral part of the chamber (so-called fluid spray). The fine particles (<100 μm) obtained by a conventional spray-drying process may, if necessary after being separated from the exhaust gas flow, be fed as nuclei, without further treatment, directly into the atomizing cone of the atomiser of the spray-dryer for the purpose of agglomeration with the liquid droplets of the active ingredient.

During the granulation step, the water can rapidly be removed from the solutions comprising the catalyst according to the invention, binder and further additives. It is expressly intended that agglomeration of the droplets forming in the atomising cone, or agglomeration of droplets with solid particles, will take place.

If necessary, the granules formed in the spray-dryer are removed in a continuous process, for example by a sieving operation. The fines and the oversize particles are either recycled directly to the process (without being redissolved) or are dissolved in the liquid active ingredient formulation and subsequently granulated again.

A further preparation method according to a) is a process in which the polymer is mixed with water and then the catalyst is dissolved/suspended in the polymer solution, thus forming an aqueous phase, the catalyst according to the invention being homogeneously distributed in that phase. At the same time or subsequently, the aqueous phase is dispersed in a water-immiscible liquid in the presence of a dispersion stabiliser in order that a stable dispersion is formed. The water is then removed from the dispersion by distillation, forming substantially dry particles. In those particles, the catalyst is homogeneously distributed in the polymer matrix.

The granules according to the invention are resistant to abrasion, low in dust, pourable and readily meterable. They can be added directly to a formulation, such as a detergent formulation, in the desired concentration of the catalyst according to the invention.

Where the coloured appearance of the granules in the detergent is to be suppressed, this can be achieved, for example, by embedding the granules in a droplet of a whitish meltable substance (“water-soluble wax”) or by adding a white pigment (e.g. TiO₂) to the granule formulation or, preferably, by encapsulating the granules in a melt consisting, for example, of a water-soluble wax, as described in EP-A-0 323 407, a white solid being added to the melt in order to reinforce the masking effect of the capsule.

b) The catalyst according to the invention can be dried in a separate step prior to the melt-granulation and, if necessary, dry-ground in a mill so that all the solids particles are <50 μm in size. The drying is carried out in an apparatus customary for the purpose, for example in a paddle dryer, vacuum cabinet or freeze-dryer.

The finely particulate catalyst is suspended in the molten carrier material and homogenised. The desired granules are produced from the suspension in a shaping step with simultaneous solidification of the melt. The choice of a suitable melt-granulation process is made in accordance with the desired size of granules. In principle, any process which can be used to produce granules in a particle size of from 0.1 to 4 mm is suitable. Such processes are droplet processes (with solidification on a cooling belt or during free fall in cold air), melt-prilling (gas/liquid cooling medium), and flake formation with a subsequent comminution step, the granulation apparatus being operated continuously or discontinuously.

Where the coloured appearance of the granules prepared from a melt is to be suppressed in the detergent, in addition to the catalyst it is also possible to suspend in the melt white or coloured pigments which, after solidification, impart the desired coloured appearance to the granules (e.g. titanium dioxide).

If desired, the granules can be covered with or encapsulated in an encapsulating material. Methods that come into consideration for such an encapsulation include the customary methods and also encapsulation of the granules by a melt consisting e.g. of a water-soluble wax, as described, for example, in EP-A-0 323 407, coacervation, complex coacervation and surface polymerisation.

Encapsulating materials (c) include e.g. water-soluble, water-dispersible or water-emulsifiable polymers and waxes.

As further additives (d) there come into consideration, for example, wetting agents, dust removers, water-insoluble or water-soluble dyes or pigments, and also dissolution accelerators, optical brighteners and sequestering agents.

Formulations according to the invention for the cleaning of table- and kitchen-ware can be used both in household dishwashers and in industrial washers. They are added by hand or using suitable metering devices. The concentrations used in the cleaning liquor are generally about from 1 to 8 g/l, preferably from 2 to 5 g/l.

A machine washing programme is generally supplemented and completed by a number of intermediate rinsing cycles with clear water after the cleaning cycle and by a clear-rinsing operation with a customary rinse agent. The use of the formulations according to the invention results, after drying, in completely clean table- and kitchen-ware that is impeccable from the hygienic standpoint.

The following Examples serve to illustrate the invention but do not limit the invention thereto. Parts and percentages relate to weight, unless otherwise indicated. Temperatures are in degrees Celsius, unless otherwise indicated.

Synthesis of Compounds of the Pyrimidine Type

### EXAMPLE 1

### 4-Chloropyridine-2-carboxylic acid ethyl ester

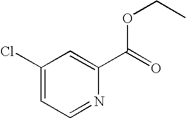 a) Step 1:

10.0 ml of (0.130 mol) of N,N-dimethylformamide are added dropwise at 40° C., with stirring, to 295 ml (4.06 mol) of thionyl chloride. Then, in the course of half an hour, 100 g (0.812 mol) of picolinic acid are added. The mixture is cautiously heated to 70° C. and stirred at that temperature for 24 hours, the gases formed being conveyed away through a wash bottle charged with sodium hydroxide solution. Concentration, and coevaporation a further three times with 100 ml of toluene each time, are carried out; the product is diluted with that solvent to 440 ml, and the solution is introduced into a mixture of 120 ml of absolute ethanol and 120 ml of toluene. The mixture is concentrated to approximately half its volume, cooled to 4° C., filtered off under suction and washed with toluene. 4-Chloropyridine-2-carboxylic acid ethyl ester hydrochloride is obtained in the form of a beige hygroscopic powder.

b) Step 2:

The hydrochloride obtained in Step 1 is taken up in 300 ml of ethyl acetate and 200 ml of deionised water and rendered neutral with 4N sodium hydroxide solution. After separation of the phases, extraction is carried out twice with 200 ml of ethyl acetate each time. The organic phases are combined, dried over sodium sulfate, filtered and concentrated. 4-Chloropyridine-2-carboxylic acid ethyl ester is obtained in the form of a brown oil which, if required, can be purified by distillation.

¹H-NMR (360 MHz, CDCl₃): 8.56 (d, J=5.0 Hz, 1H); 8.03 (d, J=1.8 Hz, 1H); 7.39 (dd, J=5.4, 1.8 Hz, 1H); 4.39 (q, J=7.0 Hz, 2H); 1.35 (t, 3H, J=7.0 Hz).

### EXAMPLE 2

### 3-(4-Chloropyrid-2-yl)-3-oxopropionic acid ethyl ester

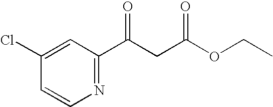

Under a nitrogen atmosphere, 4 g (approximately 60% dispersion in paraffin oil, about 100 mmol) of sodium hydride are washed twice with 60 ml of n-hexane each time, and then 400 ml of absolute tetrahydrofuran are added. The mixture is heated to 50° C. and, in the course of 2 hours, a solution of 13.36 g (72 mmol) of 4-chloropyridine-2-carboxylic acid ethyl ester and 10.04 g (114 mmol) of ethyl acetate in 60 ml of absolute tetrahydrofuran is added dropwise, during which the mixture begins to boil vigorously. When the exothermic reaction has subsided, stirring is carried out for 12 hours at room temperature to complete the reaction. The yellow suspension is poured into 400 ml of ice-water and rendered neutral with 15% hydrochloric acid, and the solution is concentrated to half its volume. Extraction is then carried out twice with 200 ml of ethyl acetate each time, and the organic extracts are combined, dried (sodium sulfate), filtered and concentrated. 14.5 g of 3-(4-chloropyrid-2-yl)-3-oxopropionic acid ethyl ester are obtained in the form of a light-brown oil, which is used for further syntheses without further purification.

¹H-NMR (360 MHz, CDCl₃): [12.33 (s, 1H, enol)]; 8.53 (d, J=5.4 Hz, 1H) [8.48 (d, J=5.4 Hz, 1H, enol)]; 8.02 (d, J=2.3 Hz, 1H) [7.88 (d, J=1.8 Hz, 1H, enol)]; 7.49-7.44 (qm, 2H) [7.35-7.30 (qm, 1H, enol)]; [6.31 (s, 1H, enol)]; 4.19-4.11 (m, 4H) [4.29-4.22 (qm, 2H, enol)]; 1.24-1.17 (tm, 3H) [1.33-1.27 (tm, 3H, enol)].

### EXAMPLE 3

### 6-(4-Chloropyrid-2-yl)-2-pyrid-2-yl-pyrimidin-4-ol (Ligand PM1)

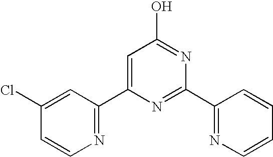

13.15 g (58 mmol) of 3-(4-chloropyrid-2-yl)-3-oxopropionic acid ethyl ester are dissolved in 400 ml of ethanol, and 9.10 g (58 mmol) of 2-amidinopyridine hydrochloride are added. After the addition of 14.44 ml of 4N sodium hydroxide solution, refluxing is carried out for 7 hours. The mixture is cooled and concentrated to a fifth of its original volume. The crude product is filtered off and recrystallised from methanol, yielding 6-(4-chloropyrid-2-yl)-2-pyrid-2-yl-pyrimidin-4-ol in the form of beige needles.

¹H-NMR (360 MHz, CDCl₃): 12.33 (br s, 1H); 8.76 (d, J=4.5 Hz, 1H); 8.69 (d, J=5.4 Hz, 1H); 8.62 (d, J=7.7 Hz, 1H); 8.50 (d, J=1.8 Hz, 1H); 8.15-8.03 (tm, 1H); 7.75-7.63 (m, 2H); 7.25 (s, 1H).

### EXAMPLE 4

### 6-[4-(4-methyl-piperazin-1-yl)-pyrid-2-yl]-2-pyrid-2-yl-pyrimidin-4-ol (Ligand PM2)

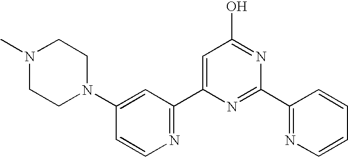

A mixture of 3.51 g (12.3 mmol) of 6-(4-chloropyrid-2-yl)-2-pyrid-2-yl-pyrimidin-4-ol, 27.4 ml (303 mmol, 20 equivalents, 30.38 g) of 1-methyl-piperazine and 84 mg (0.05 mmol, 0.05 equivalent) of zinc(II) chloride in 50 ml of 2-methyl-2-butanol is refluxed for 22 hours and concentrated to dryness using a rotary evaporator. 50 ml of water are added, 3.6 g of EDTA are added, and the pH is adjusted to 9 using dilute sodium hydroxide solution. Extraction is carried out three times using 150 ml of chloroform each time, and the organic extracts are combined and dried (sodium sulfate). Concentration is carried out using a rotary evaporator and the crude product is recrystallised from toluene. 6-[4-(4-Methyl-piperazin-1-yl)-pyrid-2-yl]-2-pyrid-2-yl-pyrimidin-4-ol is obtained in the form of a whitish solid.

¹H-NMR (360 MHz, CDCl₃): 10.99 (br s, 1H); 8.56 (d, J=4.1 Hz, 1H); 8.44 (d, J=7.7 Hz, 1H); 8.25 (d, J=5.9 Hz, 1H); 7.91-7.81 (tm, 1H); 7.78 (s, 1H); 7.48-7.33 (tm, 1H); 6.66-6.56 (m, 1H); 3.39 (t, J=5.0 Hz, 4H); 2.53 (t, J=5.0 Hz, 4H); 2.30 (s, 3H).

### EXAMPLE 5

### Quaternisation of 6-[4-(4-methyl-piperazin-1-yl)-pyrid-2-yl]-2-pyrid-2-yl-pyrimidin-4-ol with Methyl Iodide to Form Ligand PM3

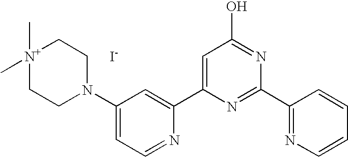

417 mg (2.94 mmol, 0.98 equivalent) of methyl iodide are added dropwise to a suspension of 1.045 g (3 mmol) of 6-[4-(4-methyl-piperazin-1-yl)-pyrid-2-yl]-2-pyrid-2-yl-pyrimidin-4-ol in 20 ml of acetonitrile. The mixture is stirred for 14 hours at room temperature, then heated to 60° C. for 10 minutes and cooled, and the resulting quaternised 6-[4-(4-methyl-piperazin-1-yl)pyrid-2-yl]-2-pyrid-2-yl-pyrimidin-4-ol is filtered off in the form of a white powder.

¹H-NMR (360 MHz, D₂O): 8.33 (d, J=4.5 Hz, 1H); 7.73-7.64 (m, 1H); 7.64-7.56 (m, 1H); 7.42-7.31 (m, 2H); 6.78 (d, 2.3 Hz, 1H); 6.33 (s, 1H); 6.31-6.26 (m, 1H).

### EXAMPLE 6

### 2,6-Di(2-pyridyl)-4-pyrimidinol (ligand PM4)

(obtainable from Bionet, Order No. 11G-917)

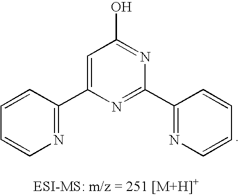

### EXAMPLE 7

### 4-Chloro-2-cyanopyridine

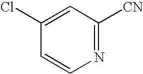

5.0 ml (0.16 equivalent) of N,N-dimethylformamide are added dropwise at 40° C., with stirring, to 150 ml (2.06 mol) of thionyl chloride. Then, in the course of half an hour, 50 g (0.406 mol) of picolinic acid are added. The mixture is cautiously heated to 70° C. and stirred at that temperature for 24 hours, the gases formed being conveyed away through a wash bottle charged with sodium hydroxide solution. Concentration, and coevaporation a further three times with 50 ml of toluene each time, are carried out. 300 ml of diethyl ether are added to the acid chloride-hydrochloride so obtained. The mixture is cooled to 0° C. using an ice/water bath, and 250 ml of 25% ammonium hydroxide solution are cautiously added. The mixture is warmed to room temperature and stirred for 16 hours to complete the reaction. Filtration is carried out, and the filter residue is boiled in 400 ml of chloroform to remove secondary products and recrystallised from 350 ml of methanol. 4-Chloro-2-picolinic acid amide is obtained in the form of a yellowish solid, which is reacted without further purification. 31.3 g (0.2 mol) of the amide obtained in that manner are suspended in 490 ml of dichloromethane and cooled to 0° C. using an ice/water bath. After the addition of 46.5 ml of N,N-dimethylformamide, 36.7 ml of phosphorus oxychloride are added dropwise in the course of 20 minutes while maintaining the temperature, and stirring is carried out for a further 6 hours with cooling. 100 ml of water are then added and the mixture is rendered neutral with 4N sodium hydroxide solution and stirred overnight at room temperature. The organic solvent is removed using a rotary evaporator, and the aqueous phase is extracted three times using 250 ml of chloroform each time. After concentrating and drying the crude product under a high vacuum, sublimation is carried out at from 70 to 90° C. and 0.2 mbar, yielding 4-chloro-2-cyanopyridine in the form of a yellowish solid.

¹H-NMR (360 MHz, CDCl₃): 8.64 (d, 5.0 Hz, 1H); 7.72 (d, J=1.8 Hz, 1H); 7.56 (dd, J=5.0, 1.8 Hz, 1H).

### EXAMPLE 8

### 2-Amidino-4-chloropyridine hydrochloride

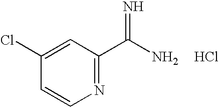

6.93 g (50 mmol) of 4-chloro-2-cyanopyridine in 40 ml of methanol are treated for one hour with 0.27 g (5 mmol) sodium methoxide. After the addition of 3.00 g (56 mmol) of ammonium chloride, refluxing is carried out for two hours. The volatile components are then removed in vacuo. The 2-amidino-4-chloropyridine hydrochloride so obtained is reacted without further purification.

¹H-NMR (360 MHz, D₂O): 8.61-8.57 (dm, 1H); 8.05 (s, 1H); 7.77-7.80 (m, 2H).

### EXAMPLE 9

### 2,6-Bis(4-chloropyrid-2-yl)-pyrimidin-4-ol (Ligand PM5)

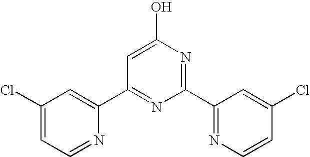

The procedure is as described in the case of 6-(4-chloropyrid-2-yl)-2-pyrid-2-yl-pyrimidin-4-ol (ligand PM1) in Example 3 except that, instead of 2-amidinopyridine hydrochloride, the 2-amidino-4-chloropyridine hydrochloride from Example 8 is used. After recrystallisation from DMSO, 2,6-bis(4-chloropyrid-2-yl)-pyrimidin-4-ol (ligand PM5) is obtained in the form of a colourless solid. ¹H-NMR (360 MHz, DMSO-d₆): 12.53 (br s, 1H); 8.74 (d, J=5.0 Hz, 1H); 8.74 (s, 1H); 8.71 (d, J=5.0 Hz, 1H); 8.64 (d, J=2.3 Hz, 1H); 7.83 (dd, J=5.0, 2.3 Hz, 1H); 7.71 (dd, J=5.0, 2.3 Hz, 1H); 7.30 (s, 1H).

### EXAMPLE 10

### 2,6-Bis[4-(4-methyl-piperazin-1-yl)-pyrid-2-yl]-pyrimidin-4-ol (Ligand PM6)

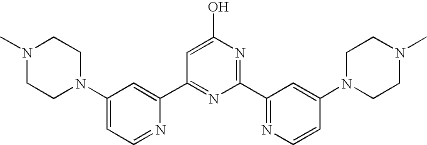

A mixture of 1.16 g (3.62 mmol), 8.04 ml (72 mmol) of N-methylpiperazine, 25 mg of zinc(II) chloride and 36 ml of 2-methyl-2-butanol is refluxed for 16 hours, cooled and filtered, and recrystallisation from 2-propanol is carried out. 2,6-bis[4-(4-methyl-piperazin-1-yl)-pyrid-2-yl]-pyrimidin-4-ol (ligand PM6) is obtained in the form of a yellowish solid.

¹H-NMR (360 MHz, DMSO-d₆): 11.92 (br s, 1H); 8.31 (d, J=5.9 Hz, 1H); 8.30 (d, J=5.9 Hz, 1H); 7.94 (br s, 2H); 7.16 (s, 1H); 7.08 (dd, J=6.3, 2.7 Hz, 1H); 6.95 8 (dd, J=6.3, 2.7 Hz, 1H); 3.52-3.41 (m, 8H); 2.54-2.49 (m, 4H); 2.48-2.43 (m, 4H); 2.24 (s, 6H).

### EXAMPLE 11

### Quaternisation of 2,6-bis[4-(4-methyl-piperazin-1-yl)-pyrid-2-yl]-pyrimidin-4-ol (Ligand PM6) with Methyl Iodide to Form Ligand PM7

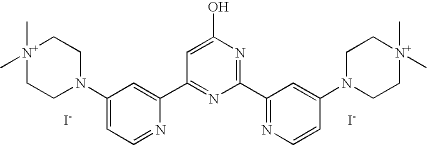

0.12 ml (1.84 mmol) of methyl iodide is added to 411 mg (0.92 mmol) of 2,6-bis[4-(4-methyl-piperazin-1-yl)-pyrid-2-yl]-pyrimidin-4-ol (ligand PM6) from Example 10 in 18 ml of acetonitrile. The mixture is stirred for 16 hours at room temperature and filtered, and the residue is washed with chloroform. The quaternised ligand PM7 is obtained in the form of a colourless solid.

¹H-NMR (360 MHz, D₂O): 8.25 (d, J=6.3 Hz, 1H); 8.19 (d, J=5.9 Hz, 1H); 7.78 (d, J=2.7 Hz, 1H); 7.50 (d, J=2.3 Hz, J=1H); 7.05 (dd, J=6.3 Hz, 2.7 Hz, 1H); 6.92 (dd, J=5.9 Hz, 2.3 Hz, 1H); 6.89 (s, 1H); 3.88-3.83 (tm, 4H); 3.81-3.76 (tm, 4H); 3.66-3.61 (m, 8H); 2.30 (s, 3H); 2.28 (s, 3H).

Synthesis of Compounds of the Triazine Type

### EXAMPLE 12

### 4,6-di-pyrid-2-yl-[1,3,5]triazin-2-ol (Ligand TZ1)

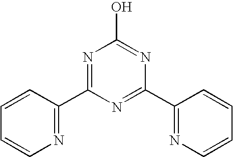

1.0 g (approximately 60% dispersion in paraffin oil, about 25 mmol) of sodium hydride is added in portions to a solution of 5.21 g (50 mmol) of 2-cyanopyridine and 1.50 g (25 mmol) of urea in 100 ml of dimethyl sulfoxide. The resulting suspension is maintained at room temperature for 3 hours and then heated at 75° C. for 23 hours, cooled and poured into 100 ml of ice-water. The mixture is rendered neutral with 2N sulfuric acid, and the crude product is filtered off and recrystallised from 55 ml of methanol, yielding 4,6-di-pyrid-2-yl-[1,3,5]triazin-2-ol in the form of a white solid.

¹H-NMR (360 MHz, CD₃OD): 8.68-8.6 (m, 4H); 7.95 (ddd, J=7.7, 7.7, 1.8 Hz, 2H); 7.50 (ddd, J=7.7, 4.5, 1.4 Hz, 2H).

### EXAMPLE 13

### 4,6-Di-pyrid-2-yl-[1,3,5]triazin-2-ylamine (Ligand TZ2)

Synthesis according to F. H. Case et al., J. Am. Chem. Soc. 1959, 81, 905-906.

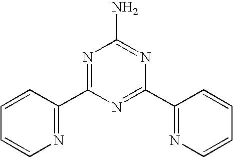

1.0 g (approximately 60% dispersion in paraffin oil, about 25 mmol) of sodium hydride is added in portions to a mixture of 5.21 g (50 mmol) of 2-cyanopyridine and 2.39 g (25 mmol) of guanidine hydrochloride in 100 ml of dimethyl sulfoxide. Stirring is carried out for 2 hours at room temperature, and then for 23 hours at 75° C. The mixture is cooled and poured into 100 ml of ice-water and filtered, yielding 4,6-di-pyrid-2-yl-[1,3,5]triazin-2-ylamine in the form of a white solid after drying in vacuo.

¹H-NMR (360 MHz, DMSO-d₆): 8.82-8.73 (md, 2H); 8.44 (d, J=8.1 Hz, 2H); 8.10-7.95 (tm, 2H); 7.90 (br s, 2H); 7.64-7.55 (m, 2H).

Synthesis of Metal Complexes

### EXAMPLE 14

### Manganese complex with 6-[4-(4-methyl-piperazin-1-yl)-pyrid-2-yl]-2-pyrid-2-yl-pyrimidin-4-ol (Ligand PM2)

503 mg (2.5 mmol) of manganese chloride tetrahydrate are added to a solution of 886 mg (2.5 mmol) of 6-[4-(4-methyl-piperazin-1-yl)-pyrid-2-yl]-2-pyrid-2-yl-pyrimidin-4-ol in 200 ml of water. The solution is then freeze-dried. C₁₉H₂₀Cl₂MnN₆O×2.92H₂O, yellow solid.

Calculated C, 43.32; H, 4.94; N, 15.95; Cl, 13.46; Mn, 10.43; H₂O, 9.98. Found C, 43.10; H, 4.95; N, 16.03; Cl, 13.29; Mn, 10.4; H₂O, 9.99.

### EXAMPLE 15

### Manganese complex with quaternised 6-[4-(4-methyl-piperazin-1-yl)-pyrid-2-yl]-2-pyrid-2-yl-pyrimidin-4-ol (Ligand PM3)

119 mg (0.6 mmol) of manganese chloride tetrahydrate are added to a solution of 294 mg (0.6 mmol) of quaternised 6-[4-(4-methyl-piperazin-1-yl)-pyrid-2-yl]-2-pyrid-2-yl-pyrimidin-4-ol in 200 ml of water. The solution is then freeze-dried. C₂₀H₂₃Cl₂MnN₆O×3.75H₂O, yellowish orange solid.

Calculated C, 35.13; H, 4.50; N, 12.29; Cl, 10.37; Mn, 8.03; H₂O, 9.88. Found C, 35.38; H 5.00; N, 12.39; Cl, 10.70; Mn, 7.99; H₂O, 9.87.

### APPLICATION EXAMPLES

### Application Example 1

Tee-stained cups were prepared according to IKW method (IKW-Arbeitskreis Maschinenspülmittel, “Methoden zur Bestimmung der Reinigungsleistung von maschinellen Geschirrspülmitteln (Part A and B)”, SÖFW, 11+14, 1998). Tea-stained cups were filled with a carbonate buffer solution (pH 9.6) containing 44 mM hydrogen peroxide and 20 μM catalyst. After 15 minutes the solution was removed, the cups were rinsed with water. The removal of the tea deposit was evaluated visually on a scale from 0 (=unchanged, very strong deposit) to 10 (=no deposit). A rating of 4 was observed in reference experiments without catalyst. Results in presence of catalysts of the present invention are summarized in the following table 1:

TABLE 1

| Mn (II) complex with ligand | Rating |
|---|---|
| PM 2 | 7 |
| PM 3 | 7.5 |
| PM 4 | 7 |
| PM 6 | 8.5 |
| PM 7 | 7.1 |

Table 1 shows that the ratings in presence of catalysts of the present invention are significantly better than the reference value.

### Application Example 2

Tea stained cups are prepared as in Application Example 1. The cups are cleaned in a automatic dishwasher at 50° C. using hard water (20° gH). In each cleaning programme 10 tea-stained cups were cleaned. The machine also contained 20 clean plates and 50 g of a food mixture as described in the IKW method as ballast. 20 g of a citrate-based formula containing 6% sodium percarbonate and 2% TAED were used as dishwasher detergent. After the cleaning operation, the removal of the tea deposit is evaluated visually on a scale from 0 (=unchanged, very strong deposit) to 10 (=no deposit). A cleaning rating of 4.5 was observed without catalyst. The addition of 1.8 μM of a Mn complex of ligand PM 6 increased the rating to 5.0.

### Application Example 3

Tea stained cups are prepared as in Application Example 1. The cups are cleaned in a automatic dishwasher at 45° C. using hard water (20° gH). In each cleaning programme 10 tea-stained cups were cleaned. The machine also contained 20 clean plates and 50 g of a food mixture as described in the IKW method as ballast. 20 g of a phosphate-based formula containing 13% sodium percarbonate were used as dishwasher detergent. Catalyst concentrations were between 75 and 100 ppm Mn (relative to the base formulation). After the cleaning operation, the removal of the tea deposit is evaluated visually on a scale from 0 (=unchanged, very strong deposit) to 10 (=no deposit). A rating of 5.8 was observed in reference experiments without catalyst. Results in presence of catalysts of the present invention are summarized in the following table 2:

TABLE 2

| catalyst | Mn content ppm | Rating |
|---|---|---|
| Mn complex of example 14 | 100 | 6.7 |
| Mn complex of ligand PM 7 | 100 | 7 |
| Mn complex of example 14 | 75 | 6.3 |
| Mn complex of ligand PM 6 | 75 | 6.5 |
| Mn complex of ligand PM 4 | 75 | 7 |

Table 2 shows that the ratings in presence of catalysts of the present invention are significantly better than the reference value.

### Application Example 4

Tea stained cups are prepared as in Application Example 1. The cups are cleaned in a automatic dishwasher at 50° C. using hard water (20° gH). In each cleaning programme 10 tea-stained cups were cleaned. The machine also contained 20 clean plates and 50 g of a food mixture as described in the IKW method as ballast. A mixture of 10 g sodium bicarbonate with 1.5 g sodium carbonate was used as dishwasher detergent. 2.6 g sodium percarbonate and 1.2 g TAED was added as basic bleach system. Catalyst concentrations were between 1.8 and 3.6 μM. After the cleaning operation, the removal of the tea deposit is evaluated visually on a scale from 0 (=unchanged, very strong deposit) to 10 (=no deposit). A cleaning rating of 4.8 was observed without catalyst. Results in presence of catalysts of the present invention are summarized in the following table 3:

TABLE 3

| | Concentration | |
|---|---|---|
| catalyst | complex (μM) | Rating |
| Mn complex of example 14 | 3.6 | 5.5 |
| Mn complex of ligand PM 6 | 1.8 | 5.4 |

Table 3 shows that the ratings in presence of catalysts of the present invention are significantly better than the reference value.

1. A method of catalyzing bleach reactions in cleaning formulations for hard surfaces comprising contacting said hard surfaces with an effective amount of a cleaning formulation comprising at least one metal complex of formula (1)
[LₙMeₘXₚ]^{z}Y_{q} (1),
wherein
Me is manganese, titanium, iron, cobalt, nickel or copper,
X is a coordinating or bridging radical,
n and m are each independently of the other an integer having a value of from 1 to 8,
p is an integer having a value of from 0 to 32,
z is the charge of the metal complex,
Y is a counter-ion,
q=z/(charge of Y), and
L is a ligand of formula (2) 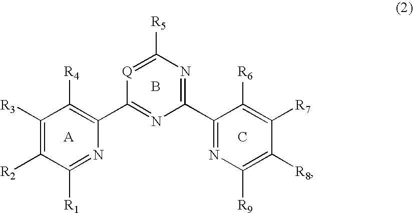
wherein
Q is N or CR₁₀,
R₁, R₂, R₃, R₄, R₅, R₆, R₇, R₈, R₉ and R₁₀ are each independently of the others hydrogen; unsubstituted or substituted C₁-C₁₈alkyl or unsubstituted or substituted aryl; cyano; halogen; nitro; —COOR₁₁ or —SO₃R₁₁ wherein
R₁₁ is in each case hydrogen, a cation or unsubstituted or substituted C₁-C₁₈alkyl or unsubstituted or substituted aryl;
—SR₁₂, —SO₂R₁₂ or —OR₁₂ wherein
R₁₂ is in each case hydrogen or unsubstituted or substituted C₁-C₁₈alkyl or unsubstituted or substituted aryl;
—NR₁₃R₁₄; —(C₁-C₆alkylene)-NR₁₃R₁₄; —N^{⊕}R₁₃R₁₄R₁₅; —(C₁-C₆alkylene)-N^{⊕}R₁₃R₁₄R₁₅; —N(R₁₂)—(C₁-C₆alkylene)-NR₁₃R₁₄; —N[(C₁-C₆alkylene)-NR₁₃R₁₄]₂; —N(R₁₂)—(C₁-C₆alkylene)-N^{⊕}R₁₃R₁₄R₁₅; —N[(C₁-C₆alkylene)-N^{⊕}R₁₃R₁₄R₁₅]₂; —N(R₁₂)—N—R₁₃R₁₄ or —N(R₁₂)—N^{⊕}R₁₃R₁₄R₁₅, wherein
R₁₃, R₁₄ and R₁₅ are each independently of the other(s) hydrogen or unsubstituted or substituted C₁-C₁₈alkyl or unsubstituted or substituted aryl, or
R₁₃ and R₁₄, together with the nitrogen atom linking them, form an unsubstituted or substituted 5-, 6- or 7-membered ring which may contain further hetero atoms.

2. A method according to claim 1, wherein Me is Mn(II) and/or Fe(II).

3. A method according to claim 1 wherein L are ligands of formula (3a) and/or (3b) 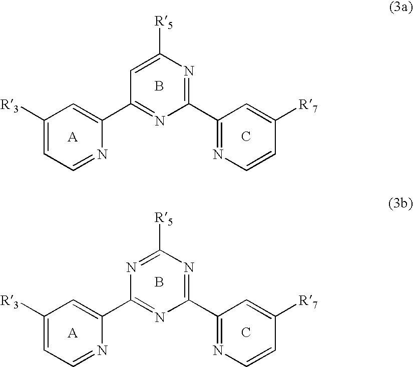
wherein
R′₅ is C₁-C₄alkoxy; hydroxy; N-mono- or N,N-di-C₁-C₄alkylamino unsubstituted or substituted by hydroxy in the alkyl moiety; or —NR₁₃R₁₄; —(C₁-C₆alkylene)-NR₁₃R₁₄; —N(R₁₂)—(C₁-C₆alkylene)-NR₁₃R₁₄; —N[(C₁-C₆alkylene)-NR₁₃R₁₄]₂; or —N(R₁₂)—N—R₁₃R₁₄, wherein
R₁₂ is hydrogen; C₁-C₁₂alkyl or unsubstituted phenyl or phenyl substituted by (substituted in the alkyl moiety by hydroxy) N-mono- or N,N-di-C₁-C₄alkylamino-, N-phenylamino-, N-naphthylamino-, phenyl-, phenoxy- or naphthyloxy, and
R₁₃ and R₁₄ are each independently of the other hydrogen, unsubstituted or hydroxy-substituted C₁-C₁₂alkyl, unsubstituted phenyl or phenyl substituted as indicated above, or
R₁₃ and R₁₄, together with the nitrogen atom linking them, form a pyrrolidine, piperidine, piperazine, morpholine or azepane ring that is unsubstituted or substituted by at least one unsubstituted C₁-C₄alkyl and/or substituted C₁-C₄alkyl, and
R′₃ and R′₇ are each independently of the other hydrogen; C₁-C₄alkoxy; hydroxy; N-mono- or N,N-di-C₁-C₄alkylamino substituted by hydroxy in the alkyl moiety; or —NR₁₃R₁₄; —(C₁-C₆alkylene)-NR₁₃R₁₄; —N(R₁₂)—(C₁-C₆alkylene)-NR₁₃R₁₄; —N[(C₁-C₆alkylene)-NR₁₃R₁₄]₂; or —N(R₁₂)—N—R₁₃R₁₄, wherein
R₁₂ is hydrogen; C₁-C₁₂alkyl or unsubstituted phenyl or phenyl substituted by (substituted in the alkyl moiety by hydroxy) N-mono- or N,N-di-C₁-C₄alkylamino-, N-phenylamino-, N-naphthylamino-, phenyl-, phenoxy- or naphthyloxy, and
R₁₃ and R₁₄ are each independently of the other hydrogen; unsubstituted or hydroxy-substituted C₁-C₁₂alkyl, unsubstituted phenyl or phenyl substituted as indicated above, or
R₁₃ and R₁₄, together with the nitrogen atom linking them, form a pyrrolidine, piperidine, piperazine, morpholine or azepane ring that is unsubstituted or substituted by at least one unsubstituted C₁-C₄alkyl and/or substituted C₁-C₄alkyl.

4. A method according to claim 1 wherein at least one Mn(II)-complex of formula (3c) and/or (3d) 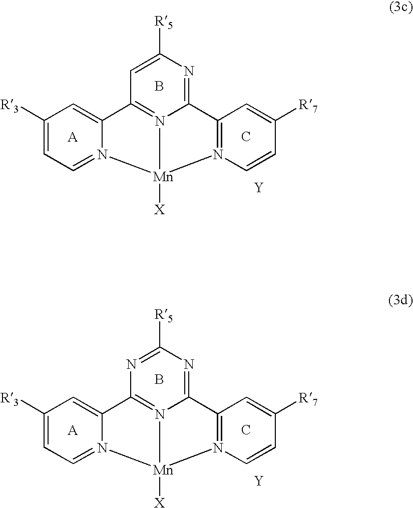
wherein
R′₅ is hydroxy; N-mono- or N,N-di-C₁-C₂alkylamino unsubstituted or substituted by hydroxy in the alkyl moiety; or —NR₁₃R₁₄; —(C₁-C₂alkylene)-NR₁₃R₁₄; —N(R₁₂)—(C₁-C₂alkylene)-NR₁₃R₁₄; —N[(C₁-C₂alkylene)-NR₁₃R₁₄]₂; or —N(R₁₂)—N—R₁₃R₁₄, wherein
R₁₂ is hydrogen; C₁-C₄alkyl or unsubstituted phenyl or phenyl substituted by (substituted in the alkyl moiety by hydroxy) N-mono- or N,N-di-C₁-C₂alkylamino-, N-phenylamino-, N-naphthylamino-, phenyl-, phenoxy- or naphthyloxy, and
R₁₃ and R₁₄ are each independently of the other hydrogen, unsubstituted or hydroxy-substituted C₁-C₄alkyl, unsubstituted phenyl or phenyl substituted as indicated above, or
R₁₃ and R₁₄, together with the nitrogen atom linking them, form a pyrrolidine, piperidine, piperazine, morpholine or azepane ring that is unsubstituted or substituted by at least one unsubstituted C₁-C₄alkyl and/or substituted C₁-C₄alkyl, and
R′₃ and R′₇ are each independently of the other hydrogen; halogen; hydroxy; N-mono- or N,N-di-C₁-C₂alkylamino substituted by hydroxy in the alkyl moiety; or —NR₁₃R₁₄; —(C₁-C₂alkylene)-NR₁₃R₁₄; —N(R₁₂)—(C₁-C₂alkylene)-NR₁₃R₁₄; —N[(C₁-C₂alkylene)-NR₁₃R₁₄]₂; or —N(R₁₂)—N—R₁₃R₁₄, wherein
R₁₂ is hydrogen; C₁-C₄alkyl or unsubstituted phenyl or phenyl substituted by (substituted in the alkyl moiety by hydroxy) N-mono- or N,N-di-C₁-C₂alkylamino-, N-phenylamino-, N-naphthylamino-, phenyl-, phenoxy- or naphthyloxy, and
R₁₃ and R₁₄ are each independently of the other hydrogen; unsubstituted or hydroxy-substituted C₁-C₄alkyl, unsubstituted phenyl or phenyl substituted as indicated above, or
R₁₃ and R₁₄, together with the nitrogen atom linking them, form a pyrrolidine, piperidine, piperazine, morpholine or azepane ring that is unsubstituted or substituted by at least one unsubstituted C₁-C₄alkyl and/or substituted C₁-C₄alkyl,
X is F⁻; Cl⁻; Br⁻; HOO⁻; ⁻CH₃COO⁻; HCOO⁻ or HO⁻, and
Y is CH₃COO⁻; HCOO⁻; ClO₄⁻; BF₄⁻; PF₆⁻; HSO₃⁻; HSO₄⁻; NO₃⁻; F⁻; Cl⁻; Br⁻ or I⁻ is used.

5. A method according to claim 4
wherein
R′₅ is hydroxy; N-mono- or N,N-di-C₁-C₂alkylamino unsubstituted or substituted by hydroxy in the alkyl moiety; or —NH₂
R₁₃ and R₁₄ are each independently of the other hydrogen, unsubstituted or
R′₃ and R′₇ are each independently of the other hydrogen; Cl; hydroxy; N-mono- or N,N-di-C₁-C₂alkylamino substituted by hydroxy in the alkyl moiety; 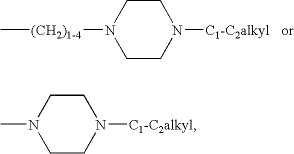
X is F⁻; Cl⁻; Br⁻; HOO⁻; CH₃COO⁻; HCOO⁻ or HO⁻, and
Y is CH₃COO⁻; HCOO⁻; ClO₄⁻; BF₄⁻; PF₆⁻; HSO₃⁻; HSO₄⁻; NO₃⁻; F⁻; Cl⁻; Br⁻ or I⁻.

6. A method according to claim 1 wherein at least one metal complex compound of formula (1′)
[L′ₙMeₘXₚ]^{z}Y_{q} (1′),
wherein
Me is manganese, titanium, iron, cobalt, nickel or copper,
X is a coordinating or bridging radical,
n and m are each independently of the other an integer having a value of from 1 to 8,
p is an integer having a value of from 0 to 32,
z is the charge of the metal complex,
Y is a counter-ion,
q=z/(charge of Y), and
L′ is a ligand of formula (2′) 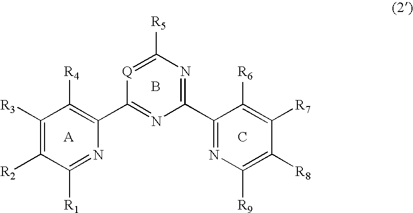
wherein
Q is N or CR₁₀,
R₁, R₂, R₃, R₄, R₅, R₆, R₇, R₈, R₉ and R₁₀ are each independently of the others hydrogen; unsubstituted or substituted C₁-C₁₈alkyl or unsubstituted or substituted aryl; cyano; halogen; nitro; —COOR₁₁ or —SO₃R₁₁ wherein
R₁₁ is in each case hydrogen, a cation or unsubstituted or substituted C₁-C₁₈alkyl or unsubstituted or substituted aryl;
—SR₁₂, —SO₂R₁₂ or —OR₁₂ wherein
R₁₂ is in each case hydrogen or unsubstituted or substituted C₁-C₁₈alkyl or unsubstituted or substituted aryl;
—NR₁₃R₁₄; —(C₁-C₆alkylene)-NR₁₃R₁₄; —N^{⊕}R₁₃R₁₄R₁₅; —(C₁-C₆alkylene)-N^{⊕}R₁₃R₁₄R₁₅; —N(R₁₂)—(C₁-C₆alkylene)-NR₁₃R₁₄; —N[(C₁-C₆alkylene)-NR₁₃R₁₄]₂; —N(R₁₂)—(C₁-C₆alkylene)-N^{⊕}R₁₃R₁₄R₁₅; —N[(C₁-C₆alkylene)-N^{⊕}R₁₃R₁₄R₁₅]₂; —N(R₁₂)—N—R₁₃R₁₄ or —N(R₁₂)—N^{⊕}R₁₃R₁₄R₁₅, wherein
R₁₃, R₁₄ and R₁₅ are each independently of the other(s) hydrogen or unsubstituted or substituted C₁-C₁₈alkyl or unsubstituted or substituted aryl, or
R₁₃ and R₁₄, together with the nitrogen atom linking them, form an unsubstituted or substituted 5-, 6- or 7-membered ring which may contain further hetero atoms,
with the proviso that
at least one of the substituents R₁ to R₁₀ contains a quaternised nitrogen atom that is not bonded directly to one of the three rings A, B and/or C,
is used.

7. A method according to claim 6 wherein at least one Mn(II)-complex of formula (3′c) and/or (3′d) 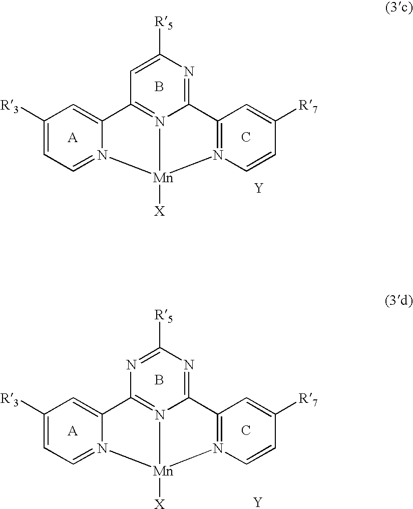
wherein
R′₅ is 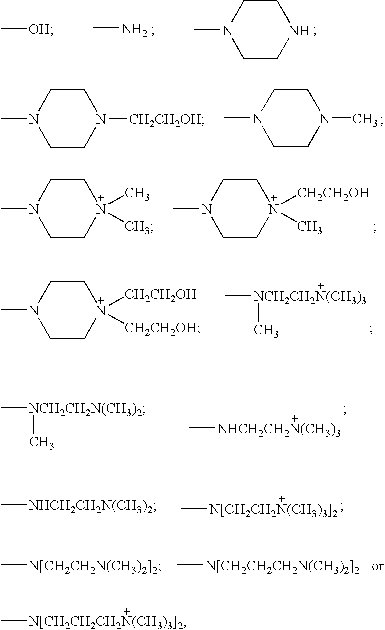
R′₃ and R′₇ are independently of each other are H; Cl; —OH; —NH₂; 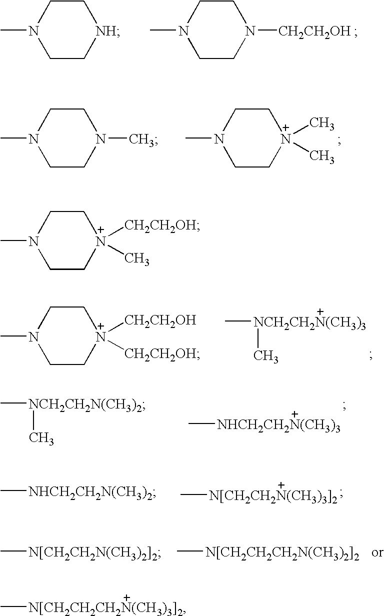
with the proviso that at least one of the substituents R′₃, R′₅ and R′₇ is 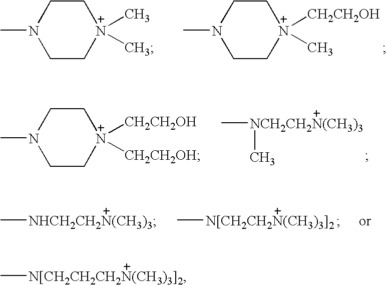
X is F⁻; Cl⁻; Br⁻; HOO⁻; CH₃COO⁻; HCOO⁻ or HO⁻, and
Y is CH₃COO⁻; HCOO⁻; ClO₄⁻; BF₄⁻; PF₆⁻; HSO₃⁻; HSO₄⁻; NO₃⁻; F⁻; Cl⁻; Br⁻ or I⁻.

8. A method according to claim 6, wherein at least one Mn(II)-complex of formula (3′c) and/or (3′d) 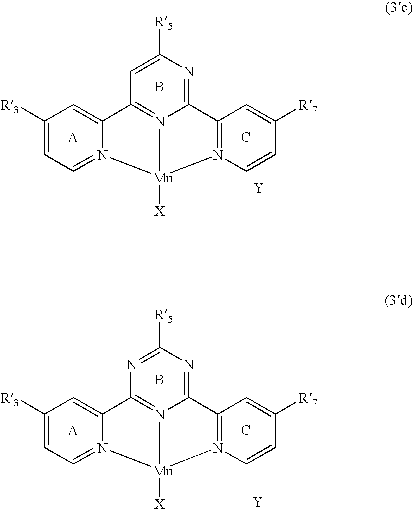
wherein
R′₅ is 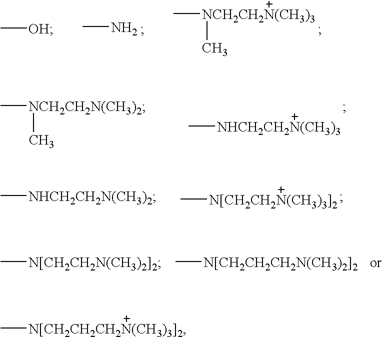
R′₃ is H; Cl; —OH; —NH₂; 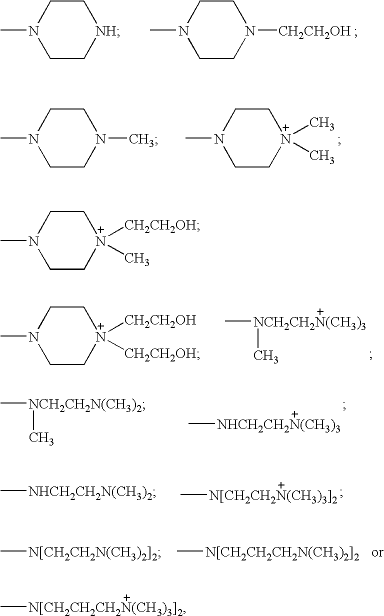
R′₇ is Cl; —OH; —NH₂; 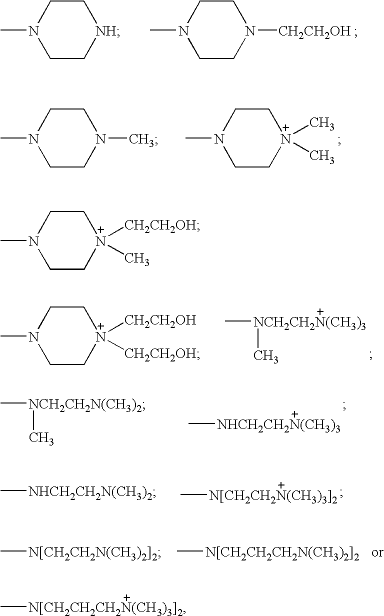
with the proviso that at least one of the substituents R′₃, and R′₇ is 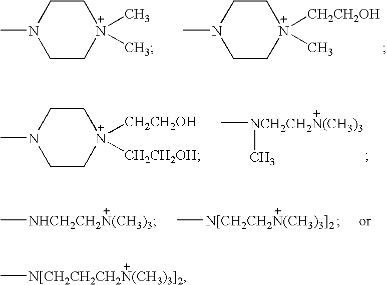
X is F⁻; Cl⁻; Br⁻; HOO⁻; CH₃COO⁻; HCOO⁻ or HO⁻, and
Y is CH₃COO⁻; HCOO⁻; ClO₄⁻; BF₄⁻; PF₆⁻; HSO₃⁻; HSO₄⁻; NO₃⁻; F⁻; Cl⁻; Br⁻ or I⁻
is used.

9. A method according to claim 6 wherein the compounds of formulae (2′), (3′c) and (3′d) one quarternised nitrogen atom is present.

10. A method according to claim 6 wherein the compounds of formulae (2′), (3′c) and (3′d) two or three quarternised nitrogen atoms are present.

11. A methods according to claim 6 wherein the compounds of formulae (2′), (3′c) and (3′d) none of the quaternised nitrogen atoms is bonded directly to one of three rings A, B and/or C.

12. A method according to any claim 1, wherein the cleaning formulations are dishwashing formulations.

13. A method according to claim 1, wherein the cleaning formulations are automatic dishwashing formulations.

14. A hard surface cleaning composition comprising at least one compound of formula (1)
[LₙMeₘXₚ]^{z}Y_{q} (1),
wherein
Me is manganese, titanium, iron, cobalt, nickel or copper,
X is a coordinating or bridging radical,
n and m are each independently of the other an integer having a value of from 1 to 8,
p is an integer having a value of from 0 to 32,
z is the charge of the metal complex,
Y is a counter-ion,
q=z/(charge of Y), and
L is a ligand of formula (2) 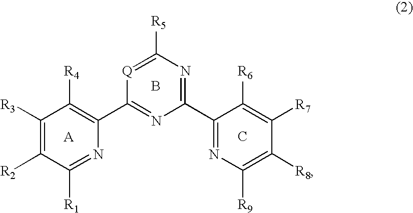
wherein
Q is N or CR₁₀,
R₁, R₂, R₃, R₄, R₅, R₆, R₇, R₈, R₉ and R₁₀ are each independently of the others hydrogen; unsubstituted or substituted C₁-C₁₈alkyl or unsubstituted or substituted aryl; cyano; halogen; nitro; —COOR₁₁ or —SO₃R₁₁ wherein
R₁₁ is in each case hydrogen, a cation or unsubstituted or substituted C₁-C₁₈alkyl or unsubstituted or substituted aryl;
—SR₁₂, —SO₂R₁₂ or —OR₁₂ wherein
R₁₂ is in each case hydrogen or unsubstituted or substituted C₁-C₁₈alkyl or unsubstituted or substituted aryl;
—NR₁₃R₁₄; —(C₁-C₆alkylene)-NR₁₃R₁₄; —N^{⊕}R₁₃R₁₄R₁₅; —(C₁-C₆alkylene)-N^{⊕}R₁₃R₁₄R₁₅; —N(R₁₂)—(C₁-C₆alkylene)-NR₁₃R₁₄; —N[(C₁-C₆alkylene)-NR₁₃R₁₄]₂; —N(R₁₂)—(C₁-C₆alkylene)-N^{⊕}R₁₃R₁₄R₁₅; —N[(C₁-C₆alkylene)-N^{⊕}R₁₃R₁₄R₁₅]₂; —N(R₁₂)—N—R₁₃R₁₄ or —N(R₁₂)—N^{⊕}R₁₃R₁₄R₁₅, wherein
R₁₃, R₁₄ and R₁₅ are each independently of the other(s) hydrogen or unsubstituted or substituted C₁-C₁₈alkyl or unsubstituted or substituted aryl, or
R₁₃ and R₁₄, together with the nitrogen atom linking them, form an unsubstituted or substituted 5-, 6- or 7-membered ring which may contain further hetero atoms.

15. A hard surface cleaning composition according to claim 14, wherein Me is Mn(II) and/or Fe(II).

16. A hard surface cleaning composition according to claim 14 wherein L are ligands of formula (3a) and/or (3b) 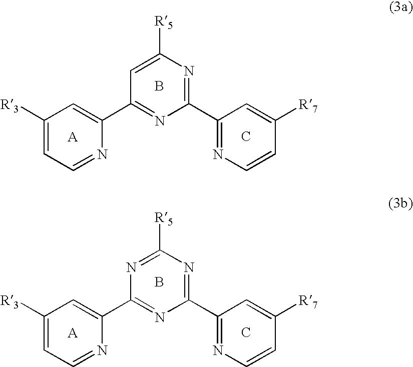
wherein
R′₅ is C₁-C₄alkoxy; hydroxy; N-mono- or N,N-di-C₁-C₄alkylamino unsubstituted or substituted by hydroxy in the alkyl moiety; or —NR₁₃R₁₄; —(C₁-C₆alkylene)-NR₁₃R₁₄; —N(R₁₂)—(C₁-C₆alkylene)-NR₁₃R₁₄; —N[(C₁-C₆alkylene)-NR₁₃R₁₄]₂; or —N(R₁₂)—N—R₁₃R₁₄, wherein
R₁₂ is hydrogen; C₁-C₁₂alkyl or unsubstituted phenyl or phenyl substituted by (substituted in the alkyl moiety by hydroxy) N-mono- or N,N-di-C₁-C₄alkylamino-, N-phenylamino-, N-naphthylamino-, phenyl-, phenoxy- or naphthyloxy, and
R₁₃ and R₁₄ are each independently of the other hydrogen, unsubstituted or hydroxy-substituted C₁-C₁₂alkyl, unsubstituted phenyl or phenyl substituted as indicated above, or
R₁₃ and R₁₄, together with the nitrogen atom linking them, form a pyrrolidine, piperidine, piperazine, morpholine or azepane ring that is unsubstituted or substituted by at least one unsubstituted C₁-C₄alkyl and/or substituted C₁-C₄alkyl-, and
R′₃ and R′₇ are each independently of the other hydrogen; C₁-C₄alkoxy; hydroxy; N-mono- or N,N-di-C₁-C₄alkylamino substituted by hydroxy in the alkyl moiety; or —NR₁₃R₁₄; —(C₁-C₆alkylene)-NR₁₃R₁₄; —N(R₁₂)—(C₁-C₆alkylene)-NR₁₃R₁₄; —N[(C₁-C₆alkylene)-NR₁₃R₁₄]₂; or —N(R₁₂)—N—R₁₃R₁₄, wherein
R₁₂ is hydrogen; C₁-C₁₂alkyl or unsubstituted phenyl or phenyl substituted by (substituted in the alkyl moiety by hydroxy) N-mono- or N,N-di-C₁-C₄alkylamino-, N-phenylamino-, N-naphthylamino-, phenyl-, phenoxy- or naphthyloxy, and
R₁₃ and R₁₄ are each independently of the other hydrogen; unsubstituted or hydroxy-substituted C₁-C₁₂alkyl, unsubstituted phenyl or phenyl substituted as indicated above, or
R₁₃ and R₁₄, together with the nitrogen atom linking them, form a pyrrolidine, piperidine, piperazine, morpholine or azepane ring that is unsubstituted or substituted by at least one unsubstituted C₁-C₄alkyl and/or substituted C₁-C₄alkyl.

17. A hard surface cleaning composition according to claim 14 wherein at least one Mn(II)-complex of formula (3c) and/or (3d) 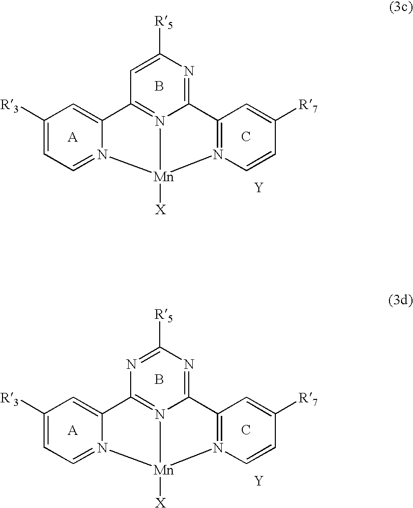
wherein
R′₅ is hydroxy; N-mono- or N,N-di-C₁-C₂alkylamino unsubstituted or substituted by hydroxy in the alkyl moiety; or —NR₁₃R₁₄; —(C₁-C₂alkylene)-NR₁₃R₁₄; —N(R₁₂)—(C₁-C₂alkylene)-NR₁₃R₁₄; —N[(C₁-C₂alkylene)-NR₁₃R₁₄]₂; or —N(R₁₂)—N—R₁₃R₁₄, wherein
R₁₂ is hydrogen; C₁-C₄alkyl or unsubstituted phenyl or phenyl substituted by (substituted in the alkyl moiety by hydroxy) N-mono- or N,N-di-C₁-C₂alkylamino-, N-phenylamino-, N-naphthylamino-, phenyl-, phenoxy- or naphthyloxy, and
R₁₃ and R₁₄ are each independently of the other hydrogen, unsubstituted or hydroxy-substituted C₁-C₄alkyl, unsubstituted phenyl or phenyl substituted as indicated above, or
R₁₃ and R₁₄, together with the nitrogen atom linking them, form a pyrrolidine, piperidine, piperazine, morpholine or azepane ring that is unsubstituted or substituted by at least one unsubstituted C₁-C₄alkyl and/or substituted C₁-C₄alkyl-, and
R′₃ and R′₇ are each independently of the other hydrogen; halogen; hydroxy; N-mono- or N,N-di-C₁-C₂alkylamino substituted by hydroxy in the alkyl moiety; or —NR₁₃R₁₄; —(C₁-C₂alkylene)-NR₁₃R₁₄; —N(R₁₂)—(C₁-C₂alkylene)-NR₁₃R₁₄; —N[(C₁-C₂alkylene)-NR₁₃R₁₄]₂; or —N(R₁₂)—N—R₁₃R₁₄, wherein
R₁₂ is hydrogen; C₁-C₄alkyl or unsubstituted phenyl or phenyl substituted by (substituted in the alkyl moiety by hydroxy) N-mono- or N,N-di-C₁-C₂alkylamino-, N-phenylamino-, N-naphthylamino-, phenyl-, phenoxy- or naphthyloxy, and
R₁₃ and R₁₄ are each independently of the other hydrogen; unsubstituted or hydroxy-substituted C₁-C₄alkyl, unsubstituted phenyl or phenyl substituted as indicated above, or
R₁₃ and R₁₄, together with the nitrogen atom linking them, form a pyrrolidine, piperidine, piperazine, morpholine or azepane ring that is unsubstituted or substituted by at least one unsubstituted C₁-C₄alkyl and/or substituted C₁-C₄alkyl,
X is F⁻; Cl⁻; Br⁻; HOO⁻; ⁻CH₃COO⁻; HCOO⁻ or HO⁻, and
Y is CH₃COO⁻; HCOO⁻; ClO₄⁻; BF₄⁻; PF₆⁻; HSO₃⁻; HSO₄⁻; NO₃⁻; F⁻; Cl⁻; Br⁻ or I⁻.

18. A hard surface cleaning composition according to claim 17
wherein
R′₅ is hydroxy; N-mono- or N,N-di-C₁-C₂alkylamino unsubstituted or substituted by hydroxy in the alkyl moiety; or —NH₂
R₁₃ and R₁₄ are each independently of the other hydrogen, unsubstituted or
R′₃ and R′₇ are each independently of the other hydrogen; Cl; hydroxy; N-mono- or N,N-di-C₁-C₂alkylamino substituted by hydroxy in the alkyl moiety; 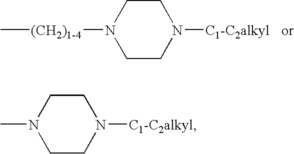
X is F⁻; Cl⁻; Br⁻; HOO⁻; CH₃COO⁻; HCOO⁻ or HO⁻, and
Y is CH₃COO⁻; HCOO⁻; ClO₄⁻; BF₄⁻; PF₆⁻; HSO₃⁻; HSO₄⁻; NO₃⁻; F⁻; Cl⁻; Br⁻ or I⁻.

19. A hard surface cleaning composition according to claim 14 wherein at least one metal complex compound of formula (1′)
[L′ₙMeₘXₚ]^{z}Y_{q} (1′),
wherein
Me is manganese, titanium, iron, cobalt, nickel or copper,
X is a coordinating or bridging radical,
n and m are each independently of the other an integer having a value of from 1 to 8,
p is an integer having a value of from 0 to 32,
z is the charge of the metal complex,
Y is a counter-ion,
q=z/(charge of Y), and
L′ is a ligand of formula (2′) 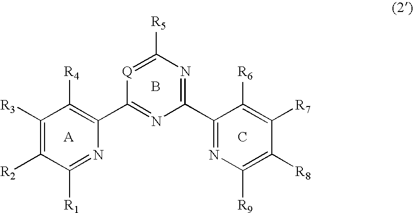
wherein
Q is N or CR₁₀,
R₁, R₂, R₃, R₄, R₅, R₆, R₇, R₈, R₉ and R₁₀ are each independently of the others hydrogen; unsubstituted or substituted C₁-C₁₈alkyl or unsubstituted or substituted aryl; cyano; halogen; nitro; —COOR₁₁ or —SO₃R₁₁ wherein
R₁₁ is in each case hydrogen, a cation or unsubstituted or substituted C₁-C₁₈alkyl or unsubstituted or substituted aryl;
—SR₁₂, —SO₂R₁₂ or —OR₁₂ wherein
R₁₂ is in each case hydrogen or unsubstituted or substituted C₁-C₁₈alkyl or unsubstituted or substituted aryl;
—NR₁₃R₁₄; —(C₁-C₆alkylene)-NR₁₃R₁₄; —N^{⊕}R₁₃R₁₄R₁₅; —(C₁-C₆alkylene)-N^{⊕}R₁₃R₁₄R₁₅; —N(R₁₂)—(C₁-C₆alkylene)-NR₁₃R₁₄; —N[(C₁-C₆alkylene)-NR₁₃R₁₄]₂; —N(R₁₂)—(C₁-C₆alkylene)-N^{⊕}R₁₃R₁₄R₁₅; —N[(C₁-C₆alkylene)-N^{⊕}R₁₃R₁₄R₁₅]₂; —N(R₁₂)—N—R₁₃R₁₄ or —N(R₁₂)—N^{⊕}R₁₃R₁₄R₁₅, wherein
R₁₃, R₁₄ and R₁₅ are each independently of the other(s) hydrogen or unsubstituted or substituted C₁-C₁₈alkyl or unsubstituted or substituted aryl, or
R₁₃ and R₁₄, together with the nitrogen atom linking them, form an unsubstituted or substituted 5-, 6- or 7-membered ring which may contain further hetero atoms,
with the proviso that
at least one of the substituents R₁ to R₁₀ contains a quaternised nitrogen atom that is not bonded directly to one of the three rings A, B and/or C,
is used.

20. A hard surface cleaning composition according to claim 19 wherein at least one Mn(II)-complex of formula (3′c) and/or (3′d) 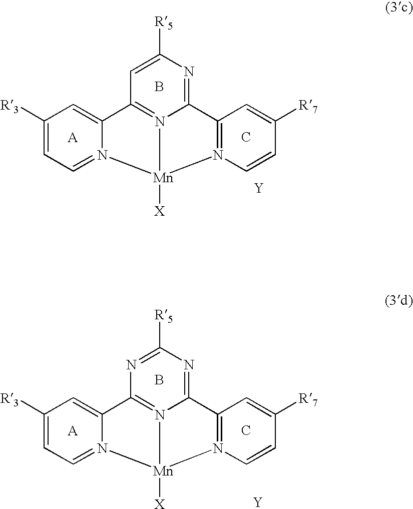
wherein
R₁₅ is 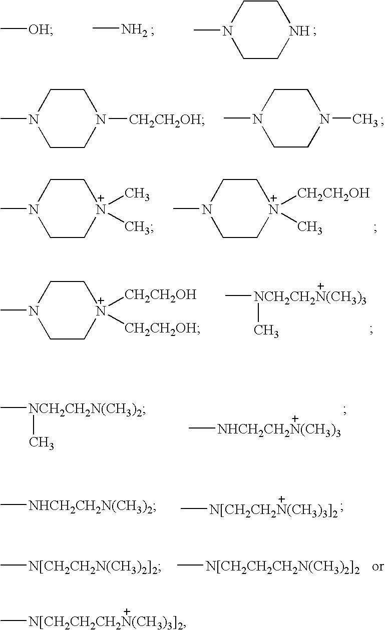
R′₃ and R′₇ are independently of each other are H; Cl; —OH; —NH₂; 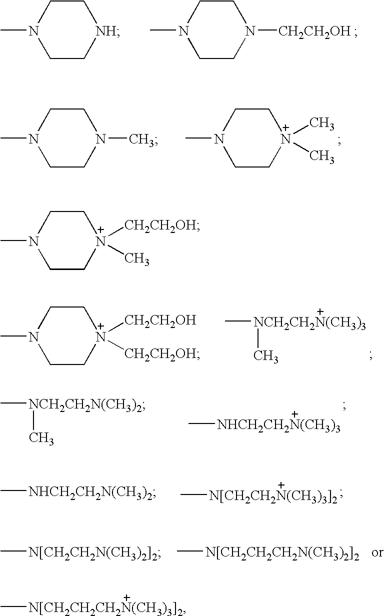
with the proviso that at least one of the substituents R′₃, R′₅ and R′₇ is 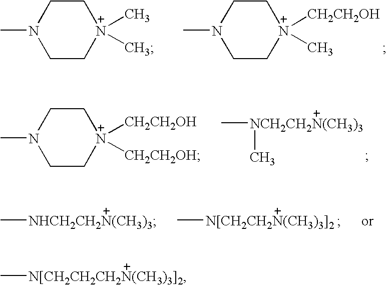
X is F⁻; Cl⁻; Br⁻; HOO⁻; CH₃COO⁻; HCOO⁻ or HO⁻, and
Y is CH₃COO⁻; HCOO⁻; ClO₄⁻; BF₄⁻; PF₆⁻; HSO₃⁻; HSO₄⁻; NO₃⁻; F⁻; Cl⁻; Br⁻ or I⁻.

21. A hard surface cleaning composition according to claim 19, wherein at least one Mn(II)-complex of formula (3′c) and/or (3′d) 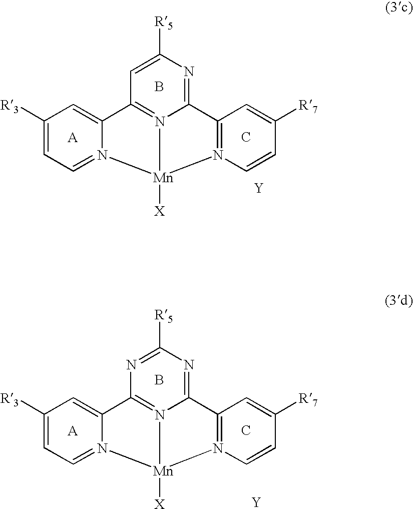
wherein
R′₅ is 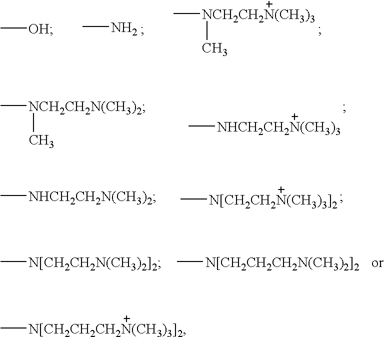
R′₃ is H; Cl; —OH; —NH₂; 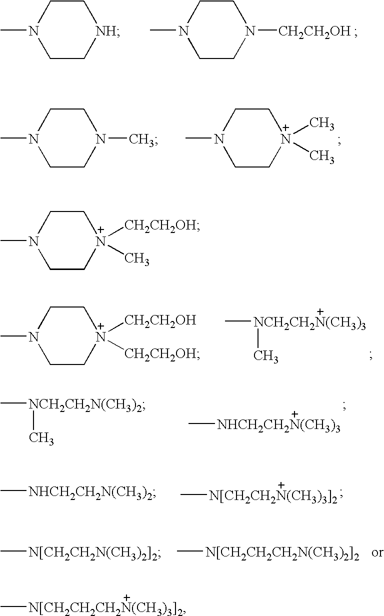
R′₇ is Cl; —OH; —NH₂; 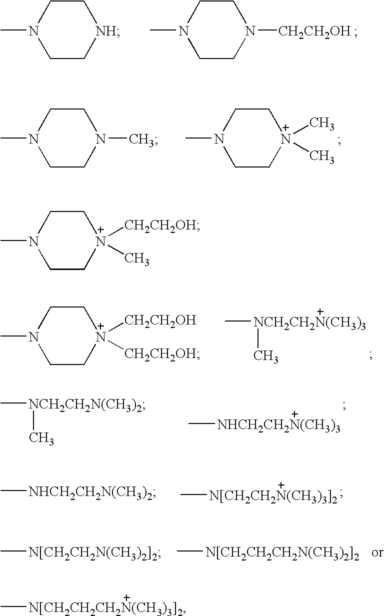
with the proviso that at least one of the substituents R′₃, and R′₇ is 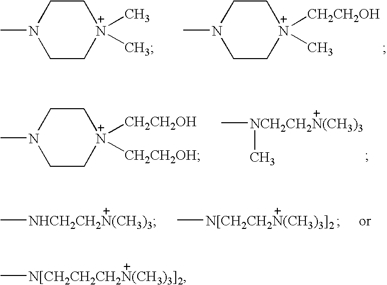
X is F⁻; Cl⁻; Br⁻; HOO⁻; CH₃COO⁻; HCOO⁻ or HO⁻, and
Y is CH₃COO⁻; HCOO⁻; ClO₄⁻; BF₄⁻; PF₆⁻; HSO₃⁻; HSO₄⁻; NO₃⁻; F⁻; Cl⁻; Br⁻ or I⁻
is used.

22. A hard surface cleaning composition according to claim 20 wherein the compounds of formulae (2′), (3′c) and (3′d) one quarternised nitrogen atom is present.

23. A hard surface cleaning composition according to claim 20 wherein the compounds of formulae (2′), (3′c) and (3′d) two or three quaternised nitrogen atoms are present.

24. A hard surface cleaning composition according to claim 19 wherein the compounds of formulae (2′), (3′c) and (3′d) none of the quaternised nitrogen atoms is bonded directly to one of three rings A, B and/or C.

25. A hard surface cleaning composition according to claim 14 wherein said composition is a dishwashing composition.

26. A hard surface cleaning composition according to claim 14 wherein said composition is an automatic dishwasher composition.
